(19) United States
(12) Patent Application Publication
(10) Pub. No.: US 2021/0285006 A1
(43) Pub. Date: Sep. 16, 2021

(54) SYSTEMS AND METHODS FOR HIGH-THROUGHPUT AUTOMATED CLONAL PLANT PRODUCTION

(71) Applicant: PIONEER HI-BRED INTERNATIONAL, INC., Johnston, IA (US)

(72) Inventors: WILLIAM JAMES GORDON-KAMM, URBANDALE, IA (US); JON AARON TUCKER REINDERS, CLIVE, IA (US)

(73) Assignee: PIONEER HI-BRED INTERNATIONAL, INC., Johnston, IA (US)

(21) Appl. No.: 17/195,881

(22) Filed: Mar. 9, 2021

Related U.S. Application Data
(60) Provisional application No. 62/987,772, filed on Mar. 10, 2020.

(51) Int. Cl. C12N 15/82

(52) U.S. Cl. CPC C12N 15/8218

(57) ABSTRACT

Automated, high-throughput and multiplexed methods for clonal plant production from maternal or paternal derived cellular structures including embryos and microspores, using morphogenic factors to produce plants are disclosed. Large scale and rapid clonal propagation methods facilitate increased efficiency in producing doubled haploid plants for breeding and for trait introgression purposes.

### CROSS-REFERENCE TO RELATED APPLICATIONS

This application claims priority to United States Provisional Application No. 62/987,772, filed Mar. 10, 2020, which is hereby incorporated herein in its entirety by reference.

### FIELD

The present disclosure relates generally to plant clonal production methods.

### BACKGROUND

Plant breeding programs identify new cultivars by screening numerous plants to identify individuals with desirable characteristics. Large numbers of progeny from crosses are typically grown and evaluated, ideally across multiple years and environments, to select the plants with the most desirable characteristics.

Typical breeding methods cross two parental plants and the filial 1 hybrid (F₁ hybrid), is the first filial generation. Hybrid vigor in a commercial F₁ hybrid is observed when two parental strains, (typically inbreds), from different heterotic groups are intercrossed. Hybrid vigor, the improved or increased function of any biological quality resulting after combining the genetic contributions of its parents, is important to commercial maize seed production and these commercial hybrid performance improvements require the continued development of new inbred parental lines. Inbred line development methods, for example, in maize, use maternal (gynogenic) doubled haploid production, in which maternal haploid embryos are selected following the fertilization of the ear of a plant resultant from a first-generation cross that has been fertilized with pollen from a so-called “haploid inducer” line. Many maternal haploid embryos resultant from fertilizing a target plant with pollen from a haploid inducer line fail to regenerate into a fertile, doubled haploid plant and few, if any, in vitro tissue culture and plantlet regeneration methods propagate multiple, fertile plants from one haploid embryo. Thus, there is a need for improving methods of producing doubled haploid plants applicable to maternal gamete doubled haploids in maize.

Plant breeding would thus benefit from systems and methods for developing efficient, high-throughput, non-destructive, automated approaches to produce large-scale doubled haploid populations or other clonally propagated production of non-recombinant and recombinant inbred lines with minimal manual involvement.

### SUMMARY

In an embodiment, the present disclosure comprises high-throughput, automated methods and compositions for 1n and 2n cells and plants useful in crop breeding programs. In a further aspect, the present disclosure provides a seed from the plant produced by the methods disclosed herein.

A high-throughput, automated method of producing a population of clonal plants, the method includes providing to a population of first plant cells a morphogenic factor in a high-throughput and automated manner, wherein the population of first plant cells are derived from a plurality of distinct parental lines; eliciting a growth response in a population of second plant cells, wherein a substantial portion of the population of second plant cells does not contain an exogenous polynucleotide encoding the morphogenic factor; and regenerating, using an automated plant cell sorting and growth platform, the population of clonal plants from the population of second plant cells that do not contain the exogenous polynucleotide encoding the morphogenic factor.

In an embodiment, the population of first plant cells are cells derived from one or more haploid (1n) cells. In an embodiment, the haploid cells are microspores. In an embodiment, the haploid cells are embryos. In an embodiment, the population of first plant cells are zygogtic embryos. In an embodiment, the population of first plant cells are diploid (2n) embryos. In an embodiment, the morphogenic factor is provided as a gene expression cassette comprising: (i) a nucleotide sequence encoding a functional WUS/WOX polypeptide; or (ii) a nucleotide sequence encoding a Babyboom (BBM) polypeptide or an Ovule Development Protein 2 (ODP2) polypeptide; or (iii) a combination of (i) and (ii). In an embodiment, the genotyping and/or phenotyping analysis is performed after the provision of the morphogenic factor and before the regeneration of the plants. In an embodiment, the genotyping and/or phenotyping is non-destructive. In an embodiment, the morphogenic factor is provided as an exogenous polypeptide. In an embodiment, the morphogenic factor is an exogenous biochemical or a chemical stimulator. In an embodiment, the nucleotide sequence encodes the WUS/WOX homeobox polypeptide and the Babyboom (BBM) polypeptide or the Ovule Development Protein 2 (ODP2) polypeptide. In an embodiment, the substantial portion of the population of second plant cells are treated with a chromosome doubling agent. In an embodiment, the methods include crossing a regenerated clonal non-transgenic plant with a plant comprising a desired genotype/phenotype; and growing offspring having the desired genotype/phenotype. In an embodiment, a plant seed produced from a doubled haploid plant produced by the methods disclosed herein and any progeny derived therefrom are provided herein.

A method of high-throughput, automated analysis of clonally propagated plant cells, the method includes characterizing a large number of plant cells that comprise the population of second plant cells or any cell derived from the population of second plant cells, wherein the characterization includes data obtained from one or more genotyping and/or phenotyping experiments; predicting phenotypic performance of the second plants cells or a substantial portion thereof using a biological model based on the genotyping and/or phenotyping data of population of second plant cells that are characterized; and selecting a second plant cell from the population of second plant cells based on the predicted phenotypic performance; and regenerating a clonal plant derived from the selected second plant cell.

In an embodiment, the characterizing step includes one or more processes selected from the group consisting of: high-throughput genotyping of DNA isolated from the second plant cell or the cell derived from the second plant cell; or high-throughput measurement or detection of RNA transcripts isolated from the second plant cell or the cell derived from the second plant cell; or high-throughput measurement or detection of nucleosome abundance or densities of chromatin isolated from the second plant cell or the cell derived from the second plant cell; or high-throughput measurement or detection of post-translational modifications of histone proteins of chromatin isolated from the second plant cell or the cell derived from the second plant cell; or high-throughput measurement or detection of epigenetic modifications of DNA or RNA isolated from the second plant cell or the cell derived from the second plant cell; or high-throughput measurement or detection of protein:DNA interactions of chromatin isolated from the second plant cell or the cell derived from the second plant cell; high-throughput measurement or detection of protein:RNA interactions or complexes isolated from the second plant cell or the cell derived from the second plant cell; and a combination of the foregoing.

In an embodiment, the predicting phenotypic performance is selected from the group consisting of: using large-scale genomic data based on genotyping by DNA sequencing of the second plant cell or the cell derived from the second plant cell; or using genomic data based on genotyping by assay of the second plant cell or the cell derived from the second plant cell; or using large-scale genomic data based on a known or predicted expression state of the second plant cell or the cell derived from the second plant cell; or using large-scale genomic data based on a known or predicted chromatin state of the second plant cell or the cell derived from the second plant cell; or using large-scale genomic data based on a known or predicted epigenetic regulatory state of the second plant cell or the cell derived from the second plant cell; or using large-scale genotype imputation of shared haplotype genomic data of the second plant cell or the cell derived from the second plant cell; or using large-scalepedigree history data of the second plant cell or the cell derived from the second plant cell; and a combination of the foregoing.

In an embodiment, the second plant cell or the cell derived from the second plant cell is selected from the group consisting of callus, undifferentiated callus, immature embryos, mature embryos, immature zygotic embryos, immature cotyledon, embryonic axis, suspension culture cells, protoplasts, leaf, leaf cells, root cells, phloem cells, pollen, seeds, suspension cultures, explants, embryos, zygotic embryos, somatic embryos, embryogenic callus, meristem, somatic meristems, organogenic callus, embryos derived from mature ear-derived seed, leaf bases, leaves from mature plants, leaf tips, immature inflorescences, tassel, immature ear, silks, cotyledons, meristematic regions, cells from leaves, cells from stems, cells from roots, cells from shoots, gametophytes, sporophytes, microspores, multicellular structures (MCS), embryo-like structures; and a combination of the foregoing.

A clonally propagated population of plant seeds of the regenerated clonal plant population disclosed herein and a plant seed or any progeny resulting from crossing the regenerated clonal plant disclosed herein with another plant and growing the offspring of the cross with one or more desired traits.

A high-throughput method of producing a plurality of transgenic plants having single copy of a trait gene expression cassette, the method includes providing to a population of haploid embryos or embryo-like structures a trait gene expression cassette and a morphogenic gene expression cassette; selecting a subset of haploid embryos or haploid embryo-like structures containing the trait gene expression cassette and no morphogenic gene expression cassette, in a non-destructive manner and optionally transferring the selected haploid embryos or the selected haploid embryo-like structures to another platform using a mechanical device that is controlled by a computer; contacting the selected haploid embryos or the selected haploid embryo-like structures with a chromosome doubling agent in an automated configuration for a period sufficient to generate doubled haploid embryos or a doubled haploid embryo-like structure; and regenerating transgenic plants from the selected doubled haploid embryos or the selected doubled haploid embryo-like structures containing the trait gene expression cassette and no morphogenic gene expression cassette.

In an embodiment, the providing to the haploid embryo or the embryo-like structure comprises particle gun delivery of the trait gene expression cassette and the morphogenic gene expression cassette. In an embodiment, the providing to the haploid embryo or the embryo-like structure comprises simultaneously contacting the haploid embryo or the embryo-like structure with the trait gene expression cassette and the morphogenic gene expression cassette. In an embodiment, the providing to the haploid embryo or the embryo-like structure comprises sequentially contacting the haploid embryo or the embryo-like structure with the trait gene expression cassette and the morphogenic gene expression cassette. In an embodiment, the providing to the haploid embryo or the embryo-like structure comprises bacterial-mediated delivery of the trait gene expression cassette and the morphogenic gene expression cassette. In an embodiment, the methods include crossing a subset of the regenerated transgenic doubled haploid plants with a population of plants comprising one or more desired genotype/phenotype in a breeding program; and growing offspring having the desired genotype/phenotype, wherein the selection of the offspring is achieved by genome prediction or another predictive process.

In an embodiment, a plant seed produced by the methods disclosed herein and any progeny derived therefrom, and a plant seed and any progeny derived therefrom resulting from a cross of a regenerated transgenic plant provided with a plant comprising a desired genotype/phenotype.

A high-throughput method of producing a population of genetic recombinant plants resulting from the product of one or more meiosis events, the method comprising: providing to a plurality of 2n embryos resulting from one or more biparental crosses, a morphogenic gene expression cassette to induce somatic embryogenesis in a platform that is configurable for automated embryo isolation and treatment; exposing the 2n embryos to conditions in a large-scale setting, where such exposure is designed to occur before, during, or after somatic embryogenesis induction, thereby changing the cell fate of a substantial portion of the 2n embryos and allowing entry of the substantial portion of the 2n embryos into meiosis thereby producing a plurality of 1n embryos; selecting a subset of the plurality of the 1n embryos containing no morphogenic gene expression cassette; contacting the 1n embryos with a chromosome doubling agent for a period sufficient to generate doubled 1n embryos in a system that is configured for automated robotic handling of the 1n embryos; and regenerating the population of genetic recombinant plants from the doubled 1n embryos containing no morphogenic gene expression cassette.

In an embodiment, the methods described herein include obtaining large-scale seed or progeny thereof from genetic recombinant plants containing no morphogenic gene expression cassette. In an embodiment, the providing and/or exposing comprises particle gun delivery or other non-microbial delivery methods. In an embodiment, the particle gun delivery comprises simultaneously contacting the 2n embryo with the morphogenic gene expression cassette and a meiosis induction expression cassette. In an embodiment, the particle gun delivery comprises sequentially contacting the 2n embryo with the morphogenic gene expression cassette and a meiosis induction expression cassette. In an embodiment, the providing and/or exposing comprises bacterial-mediated delivery. In an embodiment, the meiosis induction expression cassette comprises a product of a gene or genes produced from a DNA polynucleotide, a product of a gene or genes produced from a RNA polynucleotide, a product of a gene or genes as a protein or part of a protein complex, and a combination of the foregoing. In an embodiment, the exposing step further comprises exposing the 2n embryo to a hypoxic environment in the platform or the system that is configured to handle 2n embryos in an automated setting, exposing the 2n embryos to a reducing agent at concentrations that lower the amount of reactive oxygen species in the 2n embryos, and combinations of the foregoing. In an embodiment, the hypoxic environment comprises an environment that contains less than 10% oxygen. In an embodiment, the hypoxic environment comprises an environment that contains less than 19.5 percent oxygen. In an embodiment, the hypoxic environment is created by exposing the 2n embryos to a gas. In an embodiment, the hypoxic environment is created using non-atmospheric percentages of carbon dioxide and nitrogen relative to the percentage of oxygen. In an embodiment, the reducing agent is a liquid comprising a redox-modulatory compound. In an embodiment, the redox-modulatory compound is dissolved in the liquid. In an embodiment, the redox-modulatory compound is in a solid form and is contacted with the 2n embryo.

A high-throughput, automated/semi-automated method of producing a plurality of artificial seeds, the method includes contacting a plurality of 2n embryos resulting from one or more biparental crosses with one or more T-DNA or a polynucleotide containing a morphogenic gene expression cassette and transferring to a platform or a chamber that is configured to operate in an automated or a semi-automated fashion; collecting the population of clonal somatic embryos containing no morphogenic gene expression cassette; treating the collected clonal somatic embryos with a mature embryo containing a morphogenic gene expression cassette; and, using the mature embryo to create an artificial seed.

A large-scale method for producing a population of artificial seeds, the method includes contacting a plurality of somatic embryos or 2n embryos with a T-DNA containing a morphogenic gene expression cassette; collecting a subset of clonal somatic embryos containing no morphogenic gene expression cassette; treating the clonal somatic embryos to acquire one or more mature embryos; and, using the mature embryos to create the population of artificial seeds. In an embodiment, the 2n embryo or the somatic embryos are F1 hybrids or BC1 progenies resulting from one or more biparental crosses. In an embodiment, the artificial seed is used for generating a clonal plant.

### BRIEF DESCRIPTION OF THE DRAWINGS

FIG. 1 is a schematic flow-chart illustrating some of the steps in clonal production of doubled haploid plants. Automated, high-throughput, non-destructive processes including extraction, sampling, genotyping/phenotyping, and genome editing are illustrated.

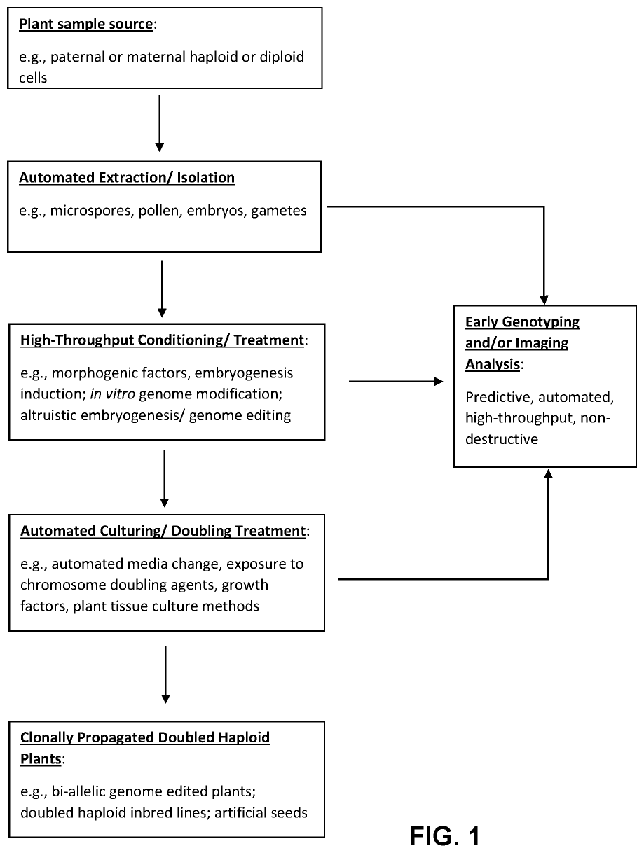

### DETAILED DESCRIPTION

The disclosures herein will be described more fully hereinafter with reference to the accompanying figures, in which some, but not all possible aspects are shown. Indeed, disclosures may be embodied in many different forms and should not be construed as limited to the aspects set forth herein; rather, these aspects are provided so that this disclosure will satisfy applicable legal requirements.

The entire content, including the drawings, specification, claims, abstract and sequence listings of International Application No. PCT/US2020/021844, filed Mar. 10, 2020 are herein incorporated by reference.

Many modifications and other aspects disclosed herein will come to mind to one skilled in the art to which the disclosed methods pertain having the benefit of the teachings presented in the following descriptions and the associated figures. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

The terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed methods belong.

The present disclosure comprises automated, high through-put methods for producing non-transgenic and transgenic plants using a morphogenic gene. The present disclosure provides methods of producing non-transgenic plants by rapidly inducing somatic embryogenesis in wild type cells using a morphogenic gene. These methods are useful in plant breeding programs, and, in crop plants. For example, in maize, the methods of the present disclosure improve the productivity of a maize maternal doubled haploid system, specifically by improving the regeneration of haploid plants per sampled haploid embryo. Due to attrition throughout the production process, haploid induction productivity under field conditions requires an average of fourteen (14) haploid embryos per doubled haploid line produced. The present disclosure provides methods of increasing productivity by producing produce multiple, clonal haploid plants from a haploid embryo.

The methods of the present disclosure are compatible with early genotyping of haploid plant cells while the cells are being cultured in vitro, thereby allowing the data outputs of these genotyping technologies to be used to predict the phenotypic performance for each plant cell genotype. This predictive selection provides the capability to design doubled haploid (DH) populations comprised of individuals with desirable genetic estimated breeding values in a manner that is expected to accelerate the rate of genetic gain relative to current breeding methods. Random selection of meiotic recombinants and the probability of recovering a superior outcome that is constrained by a relatively small number of recombinants allocated per DH population can be improved by the disclosures herein. The methods of the present disclosure permit early genotyping allowing the screening of more meiotic recombinants and facilitating the selection of candidates having the desired phenotypic/genotypic characteristics.

The methods of present disclosure provide a plant cell that is transformed with a morphogenic gene expression cassette expressing a morphogenic gene, thereby allowing the expressed morphogenic gene polypeptide to act upon cells that are not transformed during the transformation process. The methods of the present disclosure provide rapid induction of somatic embryogenesis in wild type cells in response to the activity of a translocated morphogenic gene polypeptide. The activity of a translocated morphogenic gene polypeptide in multiple cells can stimulate the development of multiple somatic embryos. The methods of the present disclosure are useful for generating multiple clonal embryos from a treated explant. When the treated explant is a maternal haploid embryo, the resulting clonal embryos can be contacted with a chromosome doubling agent to create multiple clonal doubled haploid plants per maternal haploid embryo.

The methods of the present disclosure improve productivity when used in plant breeding efforts by permitting the cross-fertilization of two individual plants derived from a common haploid embryo. This is particularly useful if flower development has shifted in the two respective plants, where one individual may be used as a pollen donor and the second as a pollen receiver, thereby allowing cross-fertilization within clones,

As used herein, the term “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. As used herein, the term “morphogenic factor” means a morphogenic gene and/or the protein expressed by a morphogenic gene.

A morphogenic gene is involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof, such as WUS/WOX genes (WUS1, WUS2, WUS3, WOX2A, WOX4, WOX5, or WOX9) see, U.S. Pat. Nos. 7,348,468 and 7,256,322 and US Patent Application Publication Numbers 2017/0121722 and 2007/0271628, herein incorporated by reference in their entirety; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39. Modulation of WUS/WOX is expected to modulate plant and/or plant tissue phenotype including plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene, MYB115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963).

Morphogenic polynucleotide sequences and amino acid sequences of WUS/WOX homeobox polypeptides are useful in the disclosed methods. As defined herein, a “functional WUS/WOX nucleotide” is any polynucleotide encoding a protein that contains a homeobox DNA binding domain, a WUS box, and an EAR repressor domain is useful in the methods of the disclosure (Ikeda et al., 2009 Plant Cell 21:3493-3505). The Wuschel protein, designated hereafter as WUS, plays a key role in the initiation and maintenance of the apical meristem, which contains a pool of pluripotent stem cells (Endrizzi, et al., (1996) Plant Journal 10:967-979; Laux, et al., (1996) Development 122:87-96; and Mayer, et al., (1998) Cell 95:805-815). Arabidopsis plants mutant for the WUS gene contain stem cells that are misspecified and that appear to undergo differentiation. WUS encodes a novel homeodomain protein which presumably functions as a transcriptional regulator (Mayer, et al., (1998) Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Constitutive expression of WUS in Arabidopsis has been shown to lead to adventitious shoot proliferation from leaves (in planta).

As used herein, the term “expression cassette” means a distinct component of vector DNA consisting of coding and non-coding sequences including 5′ and 3′ regulatory sequences that control expression in a transformed/transfected cell.

As used herein, the term “coding sequence” means the portion of DNA sequence bounded by a start and a stop codon that encodes the amino acids of a protein.

As used herein, the term “non-coding sequence” means the portions of a DNA sequence that are transcribed to produce a messenger RNA, but that do not encode the amino acids of a protein, such as 5′ untranslated regions, introns and 3′ untranslated regions. Non-coding sequence can also refer to RNA molecules such as micro-RNAs, interfering RNA or RNA hairpins, that when expressed can down-regulate expression of an endogenous gene or another transgene.

As used herein, the term “regulatory sequence” means a segment of a nucleic acid molecule which is capable of increasing or decreasing the expression of a gene. Regulatory sequences include promoters, terminators, enhancer elements, silencing elements, 5′ UTR and 3′ UTR (untranslated region).

As used herein, the term “Agro1” means an Agrobacterium harboring a plasmid with a T-DNA containing a “trait gene” expression cassette.

As used herein, the term “Agro2” means an Agrobacterium harboring a plasmid with a T-DNA containing a “morphogenic gene” expression cassette, expressing genes such as, but not limited to, BBM and/or WUS2.

As used herein, the term “transfer cassette” means a T-DNA comprising an expression cassette or expression cassettes flanked by the right border and the left border.

As used herein, the term “T-DNA” means a portion of a Ti plasmid that is inserted into the genome of a host plant cell.

As used herein, the term “embryo” means embryos and progeny of the same, immature and mature embryos, immature zygotic embryo, zygotic embryos, somatic embryos, embryogenic tissue, and embryos derived from mature ear-derived seed. An embryo is a structure that is capable of germinating to form a plant.

As used herein, the term “1n cell” means a cell containing a single set of chromosomes, typically the product of meiosis. Examples of a 1n cell include gametes such as sperm cells, egg cells, or tissues derived from a gamete through mitotic divisions, such as a 1n embryo or a 1n plant. In corn where the plant is normally diploid and the gametes are haploid, such gamete-derived embryos or plants are referred to a haploid embryos and haploid plants.

As used herein, the term “2n cell” means a cell containing two sets of chromosomes. Examples of 2n cells include a zygote, an embryo resulting from mitotic divisions of a zygote, or a plant produced by germination of a 2n embryo.

As used herein, the term “haploid plant” means a plant having a single set (genome) of chromosomes and the reduced number of chromosomes (n) in the haploid plant is equal to that in the gamete.

As used herein, the term “diploid plant” means a plant having two sets (genomes) of chromosomes and the chromosome number (2n) is equal to that in the zygote.

As used herein, the term “doubled haploid” or “doubled haploid plant” or “doubled haploid cell” means one that is developed by the doubling of a haploid set of chromosomes. A plant or seed that is obtained from a doubled haploid plant that is selfed any number of generations is still identified as a doubled haploid plant. A doubled haploid plant is considered a homozygous plant. A plant is considered a doubled haploid if it is fertile, even if the entire vegetative part of the plant does not consist of the cells with the doubled set of chromosomes. For example, a plant will be considered a doubled haploid plant if it contains viable gametes, even if the plant is chimeric.

As used herein, the term “doubled haploid embryo” means an embryo that has one or more cells containing two (2) sets of homozygous chromosomes that can then be grown into a doubled haploid plant.

As used herein, the term “clonal” means multiple propagated plant cells or plants that are genetically, epigenetically and morphologically identical.

As used herein, the term “gamete” means a 1n reproductive cell such as a sperm cell, an egg cell or an ovule cell resulting from meiosis.

As used herein, the term “haploid embryo” means a gamete-derived somatic structure.

As used herein, the term “somatic structure” means a tissue, organ or organism.

As used herein, the term “somatic cell” is a cell that is not a gamete. Somatic cells, tissues or plants can be haploid, diploid, triploid, tetraploid, hexaploid, etc. A complete set of chromosomes is referred to as being 1n (haploid), with the number of chromosomes found in a single set of chromosomes being referred to as the monoploid number (x). For example, in the diploid plant Zea mays, 2n=2x=20 total chromosomes, while in diploid rice Oryza sativa, 2n=2x=24 total chromosomes. In a triploid plant, such as banana, 2n=3x=33 total chromosomes. In hexaploid wheat Triticum aestivum, 2n=6x=42. Ploidy levels can also vary between cultivars within the same species, such as in sugarcane, Saccharum officinarum, where 2n=10x=80 chromosomes, but commercial sugarcane varieties range from 100 to 130 chromosomes.

As used herein, the term “anther” means a part of the stamen containing the microsporangia that is attached to the filament

As used herein, the term “locule” means a compartment within anthers containing the male gametes during microgametogenesis.

As used herein, the term “microgametogenesis” means the process in plant reproduction where a microgametophyte, herein called “microspores”, develops in a pollen grain to the three-celled stage of its development.

As used herein, the term “microsporangium (plural microsporangia)” means a sporangium that produces spores that give rise to male gametophytes. In nearly all land plants, sporangia are the site of meiosis and produce genetically distinct haploid spores.

As used herein, the term “microspore embryogenesis” means the activation of androgenic embryogenesis using microspores.

As used herein, the term “microspore-derived embryo” or “embryoid” or “embryo-like structure” means a cell or cells derived from a microspore with a cell fate and development characteristic of cells undergoing embryogenesis.

As used herein, the term “androgenic” means induction of androgenesis, for example, parthenogenesis in which the embryo contains only paternal chromosomes for haploid or diploid cells.

As used herein, the term “contacting”, “comes in contact with” or “in contact with” mean “direct contact” or “indirect contact”. For example, the medium comprising a doubling agent may have “direct contact” with a haploid cell or the medium comprising a doubling agent may be separated from a haploid cell by filter paper, plant tissues, or other cells, thus the doubling agent has “indirect contact” with the haploid cell and is transferred through the filter paper, plant tissues or other cells to the haploid cell.

As used herein, the term “medium” includes compounds in liquid, gas, or solid states.

As used herein, the term “selectable marker” means a transgene that when expressed in a transformed/transfected cell confers resistance to selective agents such as antibiotics, herbicides and other compounds toxic to an untransformed/untransfected cell.

Morphogenic polynucleotide sequences and amino acid sequences of Ovule Development Protein 2 (ODP2) polypeptides, and related polypeptides, e.g., Babyboom (BBM) protein family proteins are useful in the methods of the present disclosure. In an aspect, a polypeptide comprising two AP2-DNA binding domains is an ODP2, BBM2, BMN2, or BMN3 polypeptide see, US Patent Application Publication Number 2017/0121722, herein incorporated by reference in its entirety. ODP2 polypeptides useful in the methods of the present disclosure contain two predicted APETALA2 (AP2) domains and are members of the AP2 protein family (PFAM Accession PF00847). The AP2 family of putative transcription factors has been shown to regulate a wide range of developmental processes, and the family members are characterized by the presence of an AP2 DNA binding domain. This conserved core is predicted to form an amphipathic alpha helix that binds DNA. The AP2 domain was first identified in APETALA2, an Arabidopsis protein that regulates meristem identity, floral organ specification, seed coat development, and floral homeotic gene expression. The AP2 domain has now been found in a variety of proteins.

The term “plant” refers to whole plants, plant organs (e.g., leaves, stems, roots, etc.), plant tissues, plant cells, plant parts, seeds, propagules, embryos and progeny of the same. Plant cells can be differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, organogenic callus, protoplasts, embryos derived from mature ear-derived seed, leaf bases, leaves from mature plants, leaf tips, immature inflorescences, tassel, immature ear, silks, cotyledons, immature cotyledons, meristematic regions, callus tissue, cells from leaves, cells from stems, cells from roots, cells from shoots, gametophytes, sporophytes, pollen and microspores. Plant parts include differentiated and undifferentiated tissues including, but not limited to, roots, stems, shoots, leaves, pollen, seeds, tumor tissue and various forms of cells in culture (e. g., single cells, protoplasts, embryos, and callus tissue). The plant tissue may be in a plant or in a plant organ, tissue, or cell culture. Grain is intended to mean the mature seed produced by commercial growers for purposes other than growing or reproducing the species. Progeny, variants and mutants of the regenerated plants are also included within the scope of the present disclosure, provided these progeny, variants and mutants comprise the introduced polynucleotides.

The present disclosure may be used for transformation of any plant species, including, but not limited to, monocots and dicots. Monocots include, but are not limited to, barley, maize (corn), millet (e.g., pearl millet (Pennisetum glaucum), proso millet (Panicum miliaceum), foxtail millet (Setaria italica), finger millet (Eleusine coracana), teff (Eragrostis tef), oats, rice, rye, Setaria sp., sorghum, triticale, or wheat, or leaf and stem crops, including, but not limited to, bamboo, marram grass, meadow-grass, reeds, ryegrass, sugarcane; lawn grasses, ornamental grasses, and other grasses such as switchgrass and turf grass. Alternatively, dicot plants used in the present disclosure, include, but are not limited to, kale, cauliflower, broccoli, mustard plant, cabbage, pea, clover, alfalfa, broad bean, tomato, peanut, cassava, soybean, canola, alfalfa, sunflower, safflower, tobacco, Arabidopsis, or cotton.

The present disclosure also includes plants obtained by any of the disclosed methods herein. The present disclosure also includes seeds from a plant obtained by any of the disclosed methods herein. A transgenic plant is defined as a mature, fertile plant that contains a transgene.

In the disclosed methods, various plant-derived explants can be used, including immature embryos, 1-5 mm zygotic embryos, 3-5 mm embryos, and embryos derived from mature ear-derived seed, leaf bases, leaves from mature plants, leaf tips, immature inflorescences, tassel, immature ear, and silks. In an aspect, the explants used in the disclosed methods can be derived from mature ear-derived seed, leaf bases, leaves from mature plants, leaf tips, immature inflorescences, tassel, immature ear, and silks. The explant used in the disclosed methods can be derived from any of the plants described herein.

The present disclosure encompasses isolated or substantially purified nucleic acid compositions. An “isolated” or “purified” nucleic acid molecule or protein or a biologically active portion thereof is substantially free of other cellular material or components that normally accompany or interact with the nucleic acid molecule or protein as found in its naturally occurring environment or is substantially free of culture medium when produced by recombinant techniques or substantially free of chemical precursors or other chemicals when chemically synthesized. An “isolated” nucleic acid is substantially free of sequences (including protein encoding sequences) that naturally flank the nucleic acid (i.e., sequences located at the 5′ and 3′ ends of the nucleic acid) in the genomic DNA of the organism from which the nucleic acid is derived. For example, in various aspects, an isolated nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, or 1% (by dry weight) of contaminating protein. When a protein useful in the methods of the present disclosure or biologically active portion thereof is recombinantly produced, optimally culture medium represents less than about 30%, 20%, 10%, 5%, or 1% (by dry weight) of chemical precursors or non-protein-of-interest chemicals. Sequences useful in the methods of the present disclosure may be isolated from the 5′ untranslated region flanking their respective transcription initiation sites. The present disclosure encompasses isolated or substantially purified nucleic acid or protein compositions useful in the methods of the present disclosure.

Hybridization of such sequences may be carried out under stringent conditions. The terms “stringent conditions” or “stringent hybridization conditions” are intended to mean conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

The following terms are used to describe the sequence relationships between two or more nucleic acids or polynucleotides: (a) “reference sequence”, (b) “comparison window”, (c) “sequence identity”, (d) “percentage of sequence identity” and (e) “substantial identity”. As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by considering codon degeneracy, amino acid similarity, reading frame positioning and the like. Substantial identity of amino acid sequences for these purposes normally means sequence identity of at least 60%, 70%, 80%, 90% and at least 95%.

Another indication that nucleotide sequences are substantially identical is if two molecules hybridize to each other under stringent conditions. Generally, stringent conditions are selected to be about 5° C. lower than the Tm for the specific sequence at a defined ionic strength and pH. However, stringent conditions encompass temperatures in the range of about 1° C. to about 20° C. lower than the Tm, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

“Variants” is intended to mean substantially similar sequences. For polynucleotides, conservative variants include those sequences that, because of the degeneracy of the genetic code, encode the amino acid sequence of one of the morphogenic genes and/or genes/polynucleotides of interest disclosed herein. Variant polynucleotides also include synthetically derived polynucleotides, such as those generated, for example, by using site-directed mutagenesis but which still encode a protein of a morphogenic gene and/or gene/polynucleotide of interest disclosed herein. Generally, variants of a particular morphogenic gene and/or gene/polynucleotide of interest disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular morphogenic gene and/or gene/polynucleotide of interest as determined by sequence alignment programs and parameters described elsewhere herein.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present disclosure are biologically active, that is they continue to possess the desired biological activity of the native protein, that is, the polypeptide has morphogenic gene and/or gene/polynucleotide of interest activity. Such variants may result from, for example, genetic polymorphism or from human manipulation. Biologically active variants of a native morphogenic gene and/or gene/polynucleotide of interest protein disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the present disclosure may differ from that protein by as few as 1-15 amino acid residues, as few as 1-10, such as 6-10, as few as 5, as few as 4, 3, 2, or even 1 amino acid residue.

The sequences and genes disclosed herein, as well as variants and fragments thereof, are useful for the genetic engineering of plants, e.g. to produce a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

The methods of the present disclosure involve introducing a polypeptide or polynucleotide into a plant. As used herein, “introducing” means presenting to the plant the polynucleotide or polypeptide in such a manner that the sequence gains access to the interior of a cell of the plant. The methods of the present disclosure do not depend on a particular method for introducing a sequence into a plant, only that the polynucleotide or polypeptides gains access to the interior of at least one cell of the plant. Methods for introducing polynucleotide or polypeptides into plants include, stable transformation methods, transient transformation methods and virus-mediated methods.

A “stable transformation” is a transformation in which a nucleotide construct or an expression cassette introduced into a plant integrates into the genome of the plant and is capable of being inherited by the progeny thereof. “Transient transformation” means that a polynucleotide or an expression cassette is introduced into the plant and does not integrate into the genome of the plant or that a polypeptide is introduced into a plant.

Reporter genes or selectable marker genes may also be included in the expression cassettes discloses herein and used in the methods of the present disclosure.

A selectable marker comprises a DNA segment that allows one to identify or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include, but are not limited to, DNA segments that comprise restriction enzyme sites; DNA segments that encode products which provide resistance against otherwise toxic compounds (e.g., antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Selectable marker genes for selection of transformed cells or tissues can include genes that confer antibiotic resistance or resistance to herbicides. Selectable markers that confer resistance to herbicidal compounds include genes encoding resistance and/or tolerance to herbicidal compounds, such as glyphosate, sulfonylureas, glufosinate ammonium, bromoxynil, imidazolinones, and 2,4-dichlorophenoxyacetate (2,4-D).

In an aspect, the methods of the present disclosure provide transformation methods that allow positive growth selection. One skilled in the art can appreciate that conventional plant transformation methods have relied predominantly on negative selection schemes as described above, in which an antibiotic or herbicide (a negative selective agent) is used to inhibit or kill non-transformed cells or tissues, and the transgenic cells or tissues continue to grow due to expression of a resistance gene. In contrast, the methods of the present disclosure can be used with no application of a negative selective agent. Thus, although wild-type cells can grow unhindered, by comparison cells impacted by the controlled expression of a morphogenic gene can be readily identified due to their accelerated growth rate relative to the surrounding wild-type tissue. In addition to simply observing faster growth, the methods of the present disclosure provide transgenic cells that exhibit more rapid morphogenesis relative to non-transformed cells. Accordingly, such differential growth and morphogenic development can be used to easily distinguish transgenic plant structures from the surrounding non-transformed tissue, a process which is termed herein as “positive growth selection.”

The present disclosure provides methods for producing transgenic plants with increased efficiency and speed and providing significantly higher transformation frequencies and significantly more quality events (events containing one copy of a trait gene expression cassette with no vector (plasmid) backbone) in multiple inbred lines using a variety of starting tissue types, including transformed inbreds representing a range of genetic diversities and having significant commercial utility. The disclosed methods can further comprise polynucleotides that provide for improved traits and characteristics.

As used herein, “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch.

“Increased yield” of a transgenic plant of the present disclosure may be evidenced and measured in a number of ways, including test weight, seed number per plant, seed weight, seed number per unit area (i.e. seeds, or weight of seeds, per acre), bushels per acre, tons per acre, kilo per hectare. For example, maize yield may be measured as production of shelled corn kernels per unit of production area, e.g. in bushels per acre or metric tons per hectare, often reported on a moisture adjusted basis, e.g., at 15.5% moisture. Increased yield may result from improved utilization of key biochemical compounds, such as nitrogen, phosphorous and carbohydrate, or from improved tolerance to environmental stresses, such as cold, heat, drought, salt, and attack by pests or pathogens. Trait-enhancing recombinant DNA may also be used to provide transgenic plants having improved growth and development, and ultimately increased yield, as the result of modified expression of plant growth regulators or modification of cell cycle or photosynthesis pathways.

An “enhanced trait” as used in describing the aspects of the present disclosure includes improved or enhanced water use efficiency or drought tolerance, osmotic stress tolerance, high salinity stress tolerance, heat stress tolerance, enhanced cold tolerance, including cold germination tolerance, increased yield, improved seed quality, enhanced nitrogen use efficiency, early plant growth and development, late plant growth and development, enhanced seed protein, and enhanced seed oil production.

Any polynucleotide of interest or trait gene can be used in the methods of the present disclosure. Various changes in phenotype, imparted by a gene of interest or trait gene, include those for modifying the fatty acid composition in a plant, altering the amino acid content, starch content, or carbohydrate content of a plant, altering a plant's pathogen defense mechanism, altering kernel size, altering sucrose loading, and the like. The gene of interest or trait gene may also be involved in regulating the influx of nutrients, and in regulating expression of phytate genes particularly to lower phytate levels in the seed. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Heterologous coding sequences, heterologous polynucleotides, and polynucleotides of interest expressed by a promoter sequence transformed by the methods disclosed herein may be used for varying the phenotype of a plant. Various changes in phenotype are of interest including modifying expression of a gene in a plant, altering a plant's pathogen or insect defense mechanism, increasing a plant's tolerance to herbicides, altering plant development to respond to environmental stress, modulating the plant's response to salt, temperature (hot and cold), drought and the like. These results can be achieved by the expression of a heterologous nucleotide sequence of interest comprising an appropriate gene product. In specific aspects, the heterologous nucleotide sequence of interest is an endogenous plant sequence whose expression level is increased in the plant or plant part. Results can be achieved by providing for altered expression of one or more endogenous gene products, particularly hormones, receptors, signaling molecules, enzymes, transporters or cofactors or by affecting nutrient uptake in the plant. These changes result in a change in phenotype of the transformed plant. Still other categories of transgenes include genes for inducing expression of exogenous products such as enzymes, cofactors, and hormones from plants and other eukaryotes as well as prokaryotic organisms.

It is recognized that any gene of interest, polynucleotide of interest, trait gene or multiple genes/polynucleotides/traits of interest can be operably linked to a promoter or promoters and expressed in a plant transformed by the methods disclosed herein, for example insect resistance trait genes which can be stacked with one or more additional input trait genes (e.g., herbicide resistance, fungal resistance, virus resistance, stress tolerance, disease resistance, male sterility, stalk strength, and the like) or output trait genes (e.g., increased yield, modified starches, improved oil profile, balanced amino acids, high lysine or methionine, increased digestibility, improved fiber quality, drought resistance, and the like).

A promoter can be operably linked to agronomically important trait genes for expression in plants transformed by the methods disclosed herein that affect quality of grain, such as levels (increasing content of oleic acid) and types of oils, saturated and unsaturated, quality and quantity of essential amino acids, increasing levels of lysine and sulfur, levels of cellulose, and starch and protein content.

An “inducible” or “repressible” promoter can be a promoter which is under either environmental or exogenous control. Examples of environmental conditions that may affect transcription by inducible promoters include anaerobic conditions, or certain chemicals, or the presence of light. Alternatively, exogenous control of an inducible or repressible promoter can be affected by providing a suitable chemical or other agent that via interaction with target polypeptides result in induction or repression of the promoter. Inducible promoters include heat-inducible promoters, estradiol-responsive promoters, chemical inducible promoters, and the like. Pathogen inducible promoters include those from pathogenesis-related proteins (PR proteins), which are induced following infection by a pathogen; e. g., PR proteins, SAR proteins, beta-1,3-glucanase, chitinase, etc.

A “heterologous nucleotide sequence”, “heterologous polynucleotide of interest”, “heterologous polynucleotide”, or “heterologous trait gene” as used throughout the present disclosure, is a sequence that is not naturally occurring with or operably linked to a promoter. While this nucleotide sequence or trait gene is heterologous to the promoter sequence, it may be homologous or native or heterologous or foreign to the plant host. Likewise, the promoter sequence may be homologous or native or heterologous or foreign to the plant host and/or the polynucleotide of interest.

Expression modulating elements are useful in the methods of the present disclosure. “Expression modulating/modulation element” or “EME” as used herein refers to a nucleotide sequence that up or down-regulates the expression of one or more plant genes. An EME may have one or more copies of the same sequence arranged head-to-head, tail-to-head, or head-to-tail or a combination thereof of configurations. EMEs are derived from plant sequences, or from bacterial or viral enhancer elements. Expression modulating elements increase or decrease expression of operably linked nucleotide sequences.

As used herein, “vector” refers to a DNA molecule such as a plasmid, cosmid or bacterial phage for introducing a nucleotide construct, for example, an expression cassette, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants are available (Weissbach and Weissbach, (1988) In: Methods for Plant Molecular Biology, (Eds.), Academic Press, Inc., San Diego, Calif., herein incorporated by reference in its entirety). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant produced by the methods of the present disclosure containing a desired polynucleotide of interest is cultivated using methods well known to one skilled in the art.

The disclosed methods can be used to introduce into explants polynucleotides that are useful to target a specific site for modification in the genome of a plant derived from the explant. Site specific modifications that can be introduced with the disclosed methods include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, the disclosed methods can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods can be used together with a CRISPR-Cas system to provide for an effective system for modifying or altering target sites and nucleotides of interest within the genome of a plant, plant cell or seed. The Cas endonuclease gene is a plant optimized Cas9 endonuclease, wherein the plant optimized Cas9 endonuclease is capable of binding to and creating a double strand break in a genomic target sequence of the plant genome.

An “altered target site,” “altered target sequence” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Automated methods described herein are suitable to applications where a large sample number of tissues are provided, for example, in high-throughput processes, scaled screening methods, large-scale processing of transformation or tissue culture. Automation of the whole process or sub-steps thereof is accomplished, for example, by employing robotic or mechanical handling of ears or seeds. Such automation methods may rely on one or more sensors, including optical, mechanical, electrical sensors to aid in identification, positioning, extracting ears or kernels either in a single step, or in multiple separation steps. In an embodiment, the method provides substantially isolated embryos (or another clonally propagable tissue type) at a rate of between about 1000 to 100,000 or more embryos per employee-day; or between about 1000 to about 100,000, or about 1000 to about 50,000, or about 1000 to about 20,000, or about 1000 to about 10,000, or about 1000 to about 5000, or about 1000 to about 3000, or about 1000 to about 3000 embryos (or another clonally propagable tissue type) per employee-day; or between about 800 to about 100,000, or about 2000 to about 50,000, or about 2500 to about 20,000, or about 3000 to about 10,000, or about 5000 to about 15000, or about 800 to about 3000, or about 800 to about 1000 embryos (or another clonally propagable tissue type) per employee-day; or between about 2500 and about 100,000, or about 2500 to about 50,000, or about 2500 to about 20,000, or about 2500 to about 10,000, or about 2500 to about 5000, or about 2500 to about 3000 embryos (or another clonally propagable tissue type) per employee-day; or between about 5000 and about 100,000, or about 5000 to about 50,000, or about 5000 to about 20,000, or about 5000 to about 10,000 embryos (or another clonally propagable tissue type) per full-time equivalent (FTE), or any fraction or whole number in between any of the aforementioned ranges. The aforementioned ranges are also suitable for another suitable clonally propagated plant-derived material.

Methods described herein are suitable to applications where a large number of sample tissues are provided, for example, in high-throughput processes or screening, or in batch processing for genetic transformation, double haploid induction, tissue culture, seedling derived material, embryonic axes and their derived material for any type of genetic modulation. Automated methods include deploying robotic handling of the ears or seeds, pods, plants, silks, tassels, and any electro, electro mechanical application of force to any plant part, wherein those operations are controlled by one or more algorithms operable on premises or delivered through a cloud-based server operation. Automation methods described herein include sensors of various types, including optical or mechanical sensors, imaging, hyperspectral or other non-destructive analytics to aid in positioning of the tissue being used.

Automated methods of characterizing plant cells including, but not limited to, genotyping DNA isolated from a plant cell, measuring RNA transcripts isolated from a plant cell, measuring nucleosome abundance or densities of chromatin isolated from a plant cell, measuring post-translational modifications of histone proteins of chromatin isolated from a plant cell, measuring or estimating methylation status of genomic DNA, measuring epigenetic modifications of DNA or RNA isolated from a plant cell, measuring protein:DNA interactions of chromatin isolated from a plant cell, and measuring protein:RNA interactions or complexes isolated from a plant cell are useful in the methods of the present disclosure. Imaging includes, for example, shape, size, thickness of embryos or embryo-like structures obtained during the doubled-haploid process.

In certain embodiments, a system is provided for preparing an embryo or microspore derived cell sample for analysis including a receiving station, an activation station, an embryo or microspore derived cell collecting station, and a container transport mechanism. The receiving station may be configured to receive a container having a plurality of isolated compartments, each isolated compartment containing an embryo, a portion of an embryo, a clonally propagated portion of the embryo, or a microspore derived cell or cell tissue. The activation station may comprise a force applying member that includes a plurality of protrusions configured to directly contact the embryo tissue in each corresponding isolated compartment or a microfluidic device configured to compartmentalize different cell structures and optionally to break the tissue into two or more particles. The collecting station may comprise a particle directing member configured to provide an isolated passageway between each isolated compartment of the tissue (e.g., embryo or microspore or any suitable haploid tissue) and a corresponding collection cavity. The container transport mechanism may be configured to automatically move the container from the receiving station to the activating station and from the activating station to the collection station upon completion of a respective operation of the receiving station, activating station, and the collection station.

In still other embodiments, a method of preparing a representative sample (e.g., embryo or microspore or any suitable haploid tissue) for analysis is provided. The method includes receiving a sample container having at least one isolated compartment, each isolated compartment having a sample therein. In some cases, the force applying member includes at least one protrusion, and applying a force to the sample comprises pressing each protrusion into direct contact with the sample in each isolated compartment to break the sample into smaller portions, suitable for sampling.

In some embodiments, the method may also include directing the sample particles into corresponding collection cavities. The sample particles may be directed into the corresponding collection cavity of the sample collector using a directing member that includes at least one channel configured to provide an isolated passageway between each isolated compartment of the sample container and the corresponding collection cavity of the sample collector.

The characterization information and data from plant cells generated in the methods of the present disclosure is used to predict phenotypic performance of the plant cells and facilitate breeding decisions earlier in the development process. Methods of predicting phenotypic performance of plant cells including, but not limited to, using genomic data based on genotyping by DNA sequencing of a plant cell, using genomic data based on genotyping by assay of a plant cell, using genomic data based on a known or predicted expression state of a plant cell, using genomic data based on a known or predicted chromatin state of a plant cell, using genomic data based on a known or predicted epigenetic regulatory state of a plant cell, and using genotype imputation of shared haplotype genomic data of a plant cell and/or pedigree history data of a plant cell are useful in the methods of the present disclosure and facilitate the plant breeding process.

The piece of scutellum tissue may be analyzed for one or more characteristics selected from the group consisting of: a genetic marker, a single nucleotide polymorphism, a simple sequence repeat, a restriction fragment length polymorphism, a haplotype, a tag SNP, an allele of a genetic marker, a gene, a DNA-derived sequence, an RNA-derived sequence, a promoter, a 5′-untranslated region of a gene, a 3-untranslated region of a gene, microRNA, siRNA, a QTL, a satellite marker, a transgene, mRNA, ds mRNA, a transcriptional profile, a methylation pattern, and ploidy level.

Automated methods of analyzing an isolated embryo of a plant are provided herein. In such methods, a piece of cotyledon tissue or an embryo tissue is excised from an isolated embryo, wherein said excision does not cause a significant reduction in the germination potential of the embryo. A plurality of embryo samples are then analyzed in a high through-put manner for the presence or absence of one or more characteristics indicative of at least one genetic trait, through automated or semi-automated methods.

The isolated embryo tissue may be immature, and it may be canola, soybean, sunflower, alfalfa, cotton, maize, sorghum, rice, wheat, or millet. The isolated embryo may be obtained directly from a seed, or it may be derived from other tissues. The isolated embryo may be fresh or cooled. The isolated embryo is viable and is able to germinate into a plant after the embryo tissue is excised, sampled, and germinated in a high-throughput, automated manner. The isolated embryo may be of any ploidy such as but not limited to a haploid, diploid, doubled haploid, aneuploidy, tetraploid, hexaploid, or octoploid. Doubling of haploid embryos is accomplished by utilizing a chromosome doubling agent.

Excision of the clonally propagatable tissue may occur by one or more methods such as but not limited to: a drill bit, a water jet, a laser, a single blade, a set of opposing blades, a syringe, a core sampler (coring tool), a scalpel, a small diameter wire, a small diameter textured wire rope, a spatula, or a swab. Excision may be performed manually or by an automated process.

Methods of characterizing plant cells and predicting phenotypic performance can be accomplished using one or more methods disclosed in U.S. Pat. Nos. 6,399,855, 8,039,686, 8,321,147, 10,031,116, 10,102,476, US20160321396, US20170245446, US20170359978, and US20180363069 all of which are incorporated herein by reference in their entireties.

### EXAMPLES

### Example 1

### Culture Media Used in One or More High-Throughput, Cell Culturing nd/or Clonal Propagation Methods

Description of the media formations for transformation, selection and regeneration are referenced in Example 2 (See e.g., Tables 10-15) of International Application No. PCT/US2020/021844, filed Mar. 10, 2020, the contents of which are herein incorporated by reference.

The compositions of various media used in soybean transformation, tissue culture and regeneration are outlined. Medium M1 is used for initiation of suspension cultures, if this is the starting material for transformation. Media M2 and M3 represent typical co-cultivation media useful for Agrobacterium transformation of the entire range of explants listed above. Medium M4 is useful for selection (with the appropriate selective agent), M5 is used for somatic embryo maturation, and medium M6 is used for germination to produce T0 plantlets. Table 13 (wheat) and Table 14 (sorghum) of PCT/US2020/021844 are also included that describe the media used for transformation.

### Example 2

### Automated Agrobacterium-Mediated Transformation of Corn

Steps of obtaining Agrobacterium tumefaciens harboring a binary donor vector was streaked out from a −80° C. frozen aliquot onto solid 12R medium and cultured at 28° C. in the dark for 2-3 days can be automated so further picking and incubation steps are mechanized. A single colony or multiple colonies of Agrobacterium are picked from the master plate or source through electro mechanical robots and streaked onto a second plate or another platform containing 810K medium and incubated at 28° C. in the dark overnight. Agrobacterium infection medium (700A; 5 ml) and 100 mM 3′-5′-Dimethoxy-4′-hydroxyacetophenone (acetosyringone; 5 μL) are added to a capturing device under sterile conditions to make an even suspension. Optionally, the suspension can be sampled by a spectrophotometer to measure optical density (550 nm) and the master suspension can be adjusted to a reading of about 0.35-1.0, which corresponds to approximated about 0.5 to 2.0×10⁹ cfu/mL. Alternatively, Agrobacterium can be prepared for transformation by growing in liquid medium. One day before infection, a 125 ml flask is prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL) and 30 μL acetosyringone (20 mg/mL). Agrobacterium from a second plate or source is suspended into incubation receivers or container through robotic arms and placed on an orbital shaker set at 200 rpm and incubated at 28° C. overnight. The concentrated Agrobacterium are resuspended by vortex and the optical density (550 nm) of the Agrobacterium suspension is adjusted to a reading of about 0.35 to 2.0, as described above.

Ears of a maize (Zea mays L.) cultivar are surface-sterilized for 15-20 min in 20% (v/v) bleach (5.25% sodium hypochlorite) plus 1 drop of Tween 20 followed by 3 washes in sterile water in a platform that is capable of high-throughput surface sterilization of seeds in an automated or semi-automated fashion. Immature embryos (IEs) are isolated from ears through an automated mechanical device (see e.g., U.S. Pat. No. 9,307,708B2, incorporated herein by reference to the extent it relates to automated embryo extraction from maize kernels) and are placed in the Agrobacterium infection medium (700A) with acetosyringone solution. The optimal size of the embryos varies based on the inbred, but for transformation with WUS2 and ODP2 a wide size range of immature embryo sizes can be used. After collecting the embryos, the 700A medium was removed through a mechanical suction device and an appropriate amount of the Agrobacterium suspension is added to the embryos, the container is vortexed for 5-10 seconds and incubated for approximately 5 minutes under sterile conditions. The treated embryos in bulk are then transferred on 562V (or 710I) co-cultivation medium (see Example 2 of International Application No. PCT/US2020/021844, filed Mar. 10, 2020) and the excess liquid is mechanically removed using an appropriate suction device or a drain spigot. A robotic arm picks each infected embryo and is placed flat side down in a growth media platform. Optionally, the growth media platform is a plate. Each plate is incubated at 21° C. under dark conditions 1-3 days of co-cultivation. After 24 hours the treated embryos are transferred to resting medium (605J medium,) without selection through the mechanical arm that is trained to identify the treated embryos, pick them up and transfer to the resting media.

Preferentially, treated haploid embryos can be transferred to resting medium (605J medium) with a chromosome doubling (or mitotic inhibitor) agent, for example colchicine concentrations of 0.1-1.0 g/ml to cause mitotic arrest of dividing cells at metaphase by interfering with microtubule organization, for example for a 24-hour period before transfer to a resting medium (605J) medium, or preferentially 605T medium) without a chromosome doubling agent followed by incubation at 28° C. under dark conditions. Three to 7 days later, the developing embryos are transferred to maturation medium (289Q medium) without selection by a mechanized robot that can perform the transfer.

### Example 3

### High-Throughput Production of Genetically Diverse Population of Maize Haploid Embryos Through One or More Steps Involving Automation

Seed from an F1 hybrid maize plant resulting from cross fertilization of two genetically different inbred parental strains are planted and the F1 hybrid plants are used as female parent plants (pollen receivers). Genetic diversity is created per ovule, with each ovule being a unique genetic entity due to meiotic recombination during megagametogenesis. Seeds from haploid inducer lines, such as Stock 6, RWS, KEMS, KMS, ZMS, or related derivatives, or another inducer line carrying an introduced genetic modification at a genetic locus that is involved in haploid induction (e.g., maize matrilineal gene (mtl), centromeric histone 3 (CENH3)), are planted, and the resulting plants are used as male parent plants (pollen donors). The ears of the female parent plants are shoot-bagged or otherwise masked before silk emergence. The silks of the ears on the plants of the female parent plants are pollinated with viable pollen grains collected from the anthers of the male parent plants (haploid inducer plants). Both intraspecific pollination and interspecific (wide-cross) pollination are suitable for high-throughput automation as described herein.

This results in the production of about 2-30% of embryos being haploid embryos per ear, with frequencies known to differ per choice of the haploid inducer line used. Generally, at approximately 9-14 days after pollination, immature ears are harvested and the ears are surface sterilized in 30% Clorox bleach plus 0.5% Micro detergent for 20 minutes, and rinsed two times with sterile water using automated sterilization chambers and mechanical devices to collect, rotate and move the treated corn ears in a transferrable platform. Following the treatment, immature embryos are isolated for example using an automated or a semi-automated, low or high-throughput manner, from each ear.

A software-controlled electro mechanical robot is used to automate isolation of haploid embryos based on the identification of a marker in the inducer lines. Optionally, the marker is for example, a color marker that is expressed by a reporter gene. For example, if the inducer line is a stable transformed line with a fluorescent reporter gene and/or has inherited an anthocyanin reporter gene, for example the R1-scm gene (see U.S. Pat. No. 8,859,846 incorporated herein by reference in its entirety), then gene regulation by a promoter allowing the fluorescent protein and/or anthocyanin synthesis in the embryos at an early developmental stage allows ploidy determination. The identification and isolation of the haloid embryos is accomplished by coupling an image sensor with the robotic arm and applying programming logic to identify, discriminate, isolate and transfer such embryos to a different growth media or a platform.

Automated excision of a maize embryo (or another clonally propagable plant tissue) is performed through one or more mechanical elements. These can be done for example, with a mechanical blade cutter controlled by a robotic arm. Alternatively, a robotic arm can segment or separate a haploid embryo (or other clonally propagable plant tissue material) into two or more segments, which can be sampled and also grown into plants, in a high-throughput, automated fashion.

For transgenic methods, typical promoters that are useful include the maize oleosin promoter or the Zea mays Ubiquitin promoter. After paternal genome elimination, the maternal haploid embryos have only one set of chromosomes from the female parent in the embryo cells and these haploid embryos will test negative for the presence of the visible marker gene. By using this kind of visible marker, haploid embryos are identified as those without the fluorescent reporter gene and/or R1-scm expression and selected from diploid embryos expressing a fluorescent protein or anthocyanin pigmentation, which the image sensor controlled by a computing system is designed to instruct the robot to avoid picking such diploid embryos from further automated processing.

In an embodiment, a haploid inducer with a R1-scm allele stably transformed with an expression cassette encoding a ZsYELLOW fluorescent protein operably linked to the Zea mays UBIQUITIN promoter (ZmUBIpro) is used as the pollen donor and is expected to typically result in approximately 25% of the immature embryos being haploid embryos. These haploid embryos are then used in the experiments described herein that further automate the development of clonally propagated plants.

In an embodiment, delivery of a plasmid containing WUS2 improves clonal propagation of wild type, non-transformed doubled haploids and the high-throughput automation methods described herein are also applicable for such altruistic methods. For example, immature embryos of a maize F1 hybrid are crossed with a haploid inducer as described herein are transformed with Agrobacterium comprising a WUS expression cassette operably linked to a regulatory element. Following maize transformation of each haploid embryo as described, somatic embryogenesis on each treated haploid explant is activated. After approximately 6-10 days the proliferating callus tissue of each treated haploid embryo was dissected using an automated mechanical device, and each portion of dissected tissue is transferred using an automated device to maturation medium (289Q) with colchicine and cultured at 26-28° C. under dark conditions. After approximately 6-10 days the sub-cultured tissues are transferred to a light culture room at 26° C. until healthy plantlets with good roots developed. Approximately 7-14 days later, plantlets are transferred to flats containing potting soil and grown for 1 week in a growth chamber and subsequently grown for an additional 1-2 weeks in the greenhouse, and then transplanted to soil in pots and grown under greenhouse conditions. A leaf tissue sample is collected from each clonal plant in an automated way, coupled with minimal human involvement to obtain DNA, and a diagnostic PCR-based assay is performed to detect the presence/absence of the morphogenic gene expression cassette to determine the number of transgenic plants in response to treatments using each different morphogenic gene expression cassette using PCR-based methods. Additionally, isolated DNA is used for genotyping using PCR-based genetic marker assay methods. The genotypic data is used to determine allelic states inherited at genome-wide marker loci. The genotypic data is used to create a dendrogram representing the genetic distance between and within clonally-derived groups, or “clone sets”, whereby genetic distance is measured using allelic scores per assay states and are graphically plotted to represent relatedness. Within clone sets, inheritance patterns of allelic states are also examined using ideograms displaying the inheritance of parental alleles across the physical map of the ten (10) maize chromosomes.

These experiments demonstrate that “altruism” plasmids are useful for creating a population of non-transgenic, wild type haploid plants, which upon doubling, maximizes the number of selectable individuals created in a given doubled haploid (DH) population, which methods are capable of automation resulting in a high-thoughput, highly scalable maternal DH production system in plants. These methods are used to improve the effective population size of a breeding population.

### Example 4

### High-Efficiency, Large-Scale Generation of Clonal Doubled Haploid Plants in an Automated Production Facility

Approximately 8-72 hours after performing the transformation method using a morphogenic developmental protein, described in Example 3, treated haploid embryos are transferred in batch or a series of continuous mechanical robotized action onto a medium with a chromosome doubling agent, colchicine, and placed into a dark tissue culture room (28° C.). The amount of colchicine used in medium is generally 0.01%-0.2% (400-600 mg/L). Optionally, 0.05% (500 mg/L) colchicine can be used in these experiments. After 8-72 hours of chromosome doubling treatment, the treated embryos are transferred as described above and cultured using media without a chromosome doubling agent, for example to maturation medium (289Q medium). Using the methods disclosed herein, it is expected that a high percent (e.g., 60%) of haploid embryos will successfully respond to the colchicine doubling agent compared to a lower (e.g., 20-25%) as previously reported. In addition, the automated, high-throughput propagation of chromosome-doubled, clonal haploid maize embryos can result in an improved success rate of self-fertilization (fertile). Reproductive success is defined as an individual's production of seed, the D1 seed, after self-fertilization of each plant.

It is also expected that the methods of the present disclosure will provide further improvements in productivity by increasing reproductive successes, for example, by performing cross-fertilizations within clone sets when the clones reproductively complement one another. Additionally, Female Sterile, Male Sterile+Runt, and Asynchronous Flowering phenotypes can likewise be used to improve productivity by further increasing reproductive successes. Phenotyping for these agronomic parameters can be automated, thereby further increasing the efficiency of clonal production of DH plants. For example, if multiple clones are grown and such clones can reproductively complement each other, then this result indicates that within a clone set, cross fertilization between clones can further improve productivity (the production of seed) with levels higher than 70%-85% success in propagating the next generation of seed for a DH line. This result is an improvement in comparison to results of only up to 10% as previously reported.

### Example 5

### Non-Destructive, High-Throughput Early Genotyping and Selection of Doubled Haploids with Optimal Genomic Estimated Breeding Values

The following experiments demonstrate a method comprising genotyping cells derived from a clonal explant to identify, select, and propagate desirable clonal DH lines for breeding efforts. Genomic selection methods estimate the effects of genome-wide molecular markers to calculate genomic estimated breeding values (GEBVs) for individuals without phenotypes. For example, GEBV can be used as a selection criterion by predicting phenotypic performance with methods of computing predictions using genetic marker data that measure allelic states at genome-wide loci. Automated and high-throughput methods for creating genetic marker data are accomplished by sampling several DH line tissue, isolating DNA from the tissue samples, and genotyping each sample as described herein. It is therefore possible to determine allelic inhertiance patterns at genome-wide loci for computing a GEBV per DH line, using automated sample extraction, large-scale genotyping and using the GEBV prediction methods to only retain DH tissue that are desirable to producing DH plants and subsequent use thereof in a breeding program.

Breeding programs typically use DH populations of a certain size determined in part by constraints on available breeding resources, such as limited land area available for growing plants. As described in Example 4, one desirability in the current state of the art is reducing or overcoming productivity losses during DH production, for example, with losses typically up to or exceeding two-thirds of all sampled haploid embryos. An additional need is to reduce increased cost associated with genotyping more sampled haploid embryos than necessary with the assumption that these genotyped samples may likely fail to develop into fertile plants. Therefore, genotyping is typically performed in the subsequent generation—a process that includes the cost of goods for generating the entire DH population and then sampling to eliminate the plants that are needed. The disclosure herein addresses many ways to reduce such costs associated with increased genotyping and carrying-forward unwanted haploid/doubled haploid tissue into fertile plants.

As demonstrated in Example 3 where one section of the treated explant is used for DNA isolation and/or used to characterize the presence/absence of a T-DNA or another polynucleotide of interest, while other sections were used to obtain a clone set per treated haploid embryo, it is expected that such DNA can be used to obtain genetic data for computing a GEBV per DH line, preferably before growing the DH plants to maturity. In addition to the automated methods described above, sample tissue isolation from a treated explant, tranferring such isolated sample for further processing (e.g., DNA/RNA isolation or direct genotyping using the tissue itself), obtaining genotyping results, thereby sampling the treated explant to identify and eliminate unwanted explants, can be automated to increase the scale and speed of clonal DH production.

For example, a breeding cross is created using the methods as described in Example 3 and the population of treated haploid embryos is genotyped. The genetic data set is used to calculate GEBVs using methods for each treated haploid embryo being propogated, for example GEBVs for moisture, yield, plant height, and ear height. The resulting GEBVs are graphed as histogram distributions for each trait, with the count of DH lines shown in response to the predicted phenotypic values per trait. As a result, using the methods of the present disclosure, it is shown how doubled haploid populations can be characterized using GEBVs, thereby facilitating selection for further plant breeding based on GEBVs, using automated methods of tissue sampling and genotyping.

In the method of the current example, DNA extracted from sampled cells is used for genetic marker analyses. In another aspect, it is understood that sampled cells can be characterized using other methods, including but not limited to extraction of RNA, proteins, chromatin, and or metabolites to be analyzed using various methods that are available.

It is expected that enriched DH populations based on such selections using the methods of the present disclosure using automated and high-scale methods can phenotypically outperform a randomly generated population. Thus, the methods disclosed herein are expected to favorably impact the rate of genetic gain in a breeding program. In addition, the methods disclosed herein can reduce input costs when using GEBVs as a selection criterion earlier in the DH production process, thereby improving productivity of breeding programs.

### Example 6

### Automated Large-Scale Generation of Artificial Seeds or Propagules

The following experiments demonstrate clonally propagated somatic embryos created using the methods described herein are useful for creating artificial seed, for example artificial seed using clonal somatic embryos derived from an F1 hybrid cross.

F1 hybrid embryos are produced by crossing two inbred parental lines where one parent is the female parent (ear donor) and the second parent the male parent (pollen donor). Two plant varieties can be cross fertilized in a manner that prevents any outcrossing to generate F1 hybrid embryos. When the two parents are each homozygous at all loci genome wide, it is expected that the resultant embryo will be heterozygous at substantially all loci genome-wide. Automated embryo extraction methods are available to obtain induced embryos from e.g., maize ear. The methods of the present disclosure collect an immature F1 hybrid embryo for treatment as described below.

Using methods as described above to treat an F1 embryo, it is expected clonal somatic embryos will be produced. Clonal somatic embryos can be cultured to completion of the morphogenesis phase. The mature clonal somatic embryos can then be separated, for example using automated methods, including, but not limited to, equipment performing embryo extraction and/or isolation, in a manner allowing each clonal somatic embryo to be collected. An isolated clonal somatic embryo may be further treated using automated in vitro tissue culture methods to promote somatic embryo maturation resulting in the induction of seed dormancy, a quiescent state allowing somatic embryos to survive periods unfavorable for seedling germination.

For example, automated methods for producing a mature embryo can include enhancing development of the proembryo into an embryo by promoting cleavage of proembryonal cells using a modified basal medium containing a plant growth regulator selected from the group consisting of auxins, cytokinins, cyclitols and a mixture thereof. Sub-culturing a developed embryo on a modified medium containing abscisic acid and a reduced concentration of plant regulators in darkness or in a weak diffused light for 1-8 weeks inhibits further proembryogenesis. Sub-culturing the embryo on a modified basal medium in continuous light for about 7 to 8 weeks generates elongated somatic embryos. Converting elongated somatic embryos into mature embryos is performed by further culturing the elongated embryos on a modified basal medium and recovering the mature embryo using mechanized robotic methods that are fully automated or semi-automated with minimal manual intervention to select, identify, remove and transfer such maturing embryo structures.

Artificial seed is made using methods comprising encapsulating a mature somatic embryo with nutritive and protective layers to allow germination under favorable conditions like a natural seed. Such generation of artificial seeds are performed using one or more automated devices to generate layers of protective components on the mature embryo or other clonally propagated tissue material. It is expected that the artificial seed will protect the somatic embryo from mechanical damage during manipulation and transport, as well as provide nutritive support for germination of the somatic embryo to allow plant growth and development after sowing.

### Example 7

### Synchronized Generation of Clonal Doubled Haploids and Targeted Genome Modification or Site-Directed Genome Editing Through Automation

The following experiments demonstrate the stimulation of somatic embryogenesis in a plant cell by the expression of WUS2 protein in a plant cell wherein the plant cell stimulated to undergo somatic embryogenesis is not the plant cell expressing the WUS2 protein. In this method, the plant cell stimulated to undergo somatic embryogenesis is a second plant cell wherein the WUS2 protein activity is provided by a first plant cell that expresses a morphogenic gene expression cassette as described this disclosure. The second plant cell is simultaneously provided a genome modification expression cassette, thereby allowing recovery of a genome-modified, double haploid clone set. These plant cells are genome modified while containing no integrated DNA from the morphogenic gene expression cassette(s). These type of altruistic provision of morphogenic factors in a cell that is not the intended cell for purposes of helping an adjacent or a nearby cell undergo embryogenesis can be automated.

In an embodiment, immature haploid embryos of a maize F1 hybrid crossed with a haploid inducer are transformed using Agrobacterium strain LBA4404 THY. The Agrobacterium mixture is used to transform haploid embryos. The second expression cassette contains a polynucleotide expressing a functional Cas9 protein, two gRNAs for cleaving two target sites, an embryo-expressed cyan fluorescent protein, a selectable, and a maize-optimized Cre recombinase. In this construct, Cre activity is useful for excising the T-DNA polynucleotide sequence encoding the Cas9 protein, two gRNAs, and the Cre recombinase coding sequences resulting in a T-DNA conferring a cyan color marker and kanamycin resistance. Following the methods as described herein, sixteen (16) clone sets are potted in soil, wherein each clone set comprised four clonal plants per treated haploid embryo. Plants surviving transplanting to soil are used for leaf tissue sampling.

After plantlet regeneration as described in Example 3, leaf tissue is sampled and the target site is sequenced for evidence Cas-mediated gene editing. It is expected that certain mutations will be transmitted via the germline into the next generation, making this useful for obtaining a genome-modified, double haploid clone set lacking a T-DNA with a morphogenic expression cassette(s).

In vitro gene editing and clonal doubled haploid production using plasmids are produced where one or more steps are automated to improve efficiency and cost of goods. Agrobacterium strains comprising different plasmids affect both the induction of somatic embryos and the frequency of regenerated plants that had T-DNA with a morphogenic gene expression cassette(s). A stronger regulatory element increase the total number of clonal plants generated and reduce the frequencies of the plants having a stably incorporated morphogenic gene expression cassette T-DNA insertion. Alternatively, expression cassettes encoding WUS fusion proteins increase the total number of clonal plants generated and increased the frequencies of the plants having a stable morphogenic gene expression cassette T-DNA insertion.

Large-scale, automated methods show that the in-vitro treatment used herein produce a genome-modified plant without stable integration of exogenous DNA (e.g., T-DNA) encoding the gene modification apparatus. In an embodiment, such a result is achieved from transient expression of the T-DNA, thereby producing a mutation at the genomic target site. Regeneration of a plant with such a gene edit is achieved with no detection of the WUS-containing T-DNA, and thus is stimulated after being contacted by WUS protein activity. This demonstrates that a gene-edited D0 plant is derived from a plant cell that had never been stably transformed with a foreign DNA. Highly scalable methods to accomplish this altruistic genome editing through automated or semi-automated techniques provide a cost-efficient platform to generate hundreds or thousands of genome-wide edits.

It is expected that certain mutations will be transmitted via the germline into the next generation, and are useful for obtaining a genome-modified, double haploid clone set lacking a T-DNA with a morphogenic expression cassette(s). In an aspect, given a D0 plant is derived from a plant cell having been transiently contacted by only the WUS protein, Cas9 protein and gRNA(s) provided to a plant cell, the method of the present disclosure can obtain a genome-modified doubled haploid plant wherein that plant per se is obtained without the process of introducing a polynucleotide into the genome of the plant. Thus, it can be demonstrated that not only clonal propagation per se is achieved, but also the ability to obtain clonal doubled haploid plants having a targeted genome modification is possible. It is expected that the mutations will be transmitted via the germline into the next generation of plants providing a genome-modified, double haploid clone set lacking a T-DNA with a morphogenic gene expression cassette(s). It is also expected that co-expression of other such morphogenic genes, or any gene product useful for promoting embryogenesis can be used in the manner described herein to improve both clonal propagation per se, as well as the recovery of genome-modified plants lacking T-DNA integration.

The automated methods of the present disclosure demonstrate obtaining genome-modified doubled haploid plants, which represent improvements over methods of editing a haploid progeny using a pollen from a haploid inducer plant, wherein the inducer expresses a DNA modification enzyme and optionally at least one guide RNA. In contrast, using the methods described herein provide significant improvements in gene editing efficiency and the range of edits obtained. The automated methods disclosed herein for haploid induction editing technology is useful in plant breeding methods and enables direct editing of elite inbred lines by a single cross. Using the methods of the present disclosure in a breeding program saves in labor costs and time because the present methods generally do not require the evaluation and selection of all new events per construct per genome modification target each time a new genome modification is introduced into an elite breeding line or breeding population as may be required by other methods.

The automated methods disclosed herein not only improve gene-editing efficiencies, for example both SDN-1 and SDN-2 mutations are achieved at greater efficiencies than other methods, the methods of the present disclosure also surprisingly provide a genome-modified plant lacking any stably transformed foreign DNA. The methods of the present disclosure provide these results in relatively less time, with much greater agility, and with suitable scalability for meeting the unsolved needs of plant breeding programs to continue improving the phenotypic performance of elite germplasm than other methods. In addition, methods involving automated steps to introduce genome editing components into haploid tissue also demonstrate that using a gene targeting system comprising double strand breaks (DSB) at a site-specific genomic target site, or region, and homology direted repair (HDR) facilitate directional integration of a desired nucleotide sequence into corresponding homologous recombination sites of a haploid plant genome.

The methods described here allow generating an allelic series of mutations within a clone set of a doubled haploid line. In one aspect, the automated methods of the present disclosure allow for sampling and sequence verification of a modification at the target site prior to flowering, thus, providing the option to screen, select, and cross selected plants in a controlled manner and in a highly scalable fashion. For example, within a clone set, here referred to as “intra-clonal cross-breeding”, it is expected any randomly inserted T-DNA integration sites can be selected against in progeny produced from an intra-clonal F1 cross. It is expected that diagnostic assays for detecting T-DNA can achieve the identification of progeny that have inherited only wild type alleles in respect to parental T-DNA integration sites that become hemizygous in the intra-clonal F1 cross.

A second purpose for intra-clonal cross-breeding is the expectation that a gene edit can be fixed to homozygosiy upon self-fertilization of the progeny having only wild type alleles in respect to parental T-DNA integration sites. In this manner, intra-clonal cross-breeding can provide an allelic series of mutations within a clone set.

This differentiated trait introgression method offers additional benefits for trait integration relative to other methods. For example, back-crossing of a trait locus is commonly used and that method requires first creating the edit in a transformable maize line. After that line is created, that line is then used as a “donor” parent, during a series of back-crossing to a recipient line, used as a “recurrent” parent. The purpose of back-crossing is to achieve recovery of the recurrent parent genome with only the introgression of the the gene-modified target site locus from the “donor” parent. In comparison to the present disclosure, conventional back-crossing methods are more labor intensive and require more breeding time, notably as the number of parental conversions increases, for example, in comparison to an effort to introgress desired mutations into each doubled haploid line within a population as described herein. Thus, the method described here presents a novel method for “forward” breeding of a gene-modified trait at the population level.

From a quantitative genetics perspective, the ability to derive an allelic series of mutations at a target site achieves other useful advantages for studying causal genotype-to-phenotype relationships. First, an allelic series within a clone set provides one level of genetic analysis that can be performed. Second, multiple allelic series for multiple clone sets comprises a second level of genetic analysis across a breeding population, or alternatively a multitude of populations, that can be performed. Together, these results are expected to support novel plant breeding methods that are an improvement to the state of the art.

### Example 8

### Genotype-Independent, High-Throughput Automated Generation Clonal Doubled Haploids from Responsive Paternal (Microspore)-Derived Cells

The following experiments demonstrate using a negative selection system to select against transgenic cells or resulting transgenic plants thereof, to produce non-transgenic, wild type clonal doubled haploid plants derived from paternal haploid cells, in a high-throughput, automated, large-scale production system.

In this method, embryogenic growth is stimulated in non-transformed paternal cells, and particularly in a microspore-derived cell. Since the number of paternally-derived haploid cells are in large numbers, the paternal-derived cells are especially suitable for the high-throughput automated methods to isolate, segregating, tracking, and transferring clonally propagated paternal haploid cells. Further, the methods of the present disclosure are useful for generating populations of microspore-derived clonal doubled haploids.

Microspores are extracted, isolated, and purified from a donor tissue or organ producing such gametic cells using automated methods. These cells are cultured in a growth medium to induce an embryogenic response. Genotype-independent methods are used in an automated manner to stimulate improved frequency of responsive microspore-derived embryos.

In an embodiment, methods of the present disclosure are practiced using ATCC40520 corn microspores cultured in an appropriate platform suitable for automation and high-throughput efficiency, in a 9% sucrose induction medium at 28° C. under dark conditions for seven (7) to twenty-one (21) days, preferentially fourteen (14) days after initiating the culture. During initial phases of corn microspore embryogenesis, the cellular response results in repeated cell divisions, thereby forming multicellular structures (MCS). With continued development, MCS grows, resulting in embryo-like structures enclosed within the original microspore wall, known as the exine, the exine then characteristically degrades to allow further development into haploid embryos and regeneration of plantlets. These MCS are handled by an electromechanical robot controlled by a computer.

MCS are transferred from a liquid culture in an automated manner and washed three times with an appropriate volume (depending on the size/scale of the microspore production platform set-up) of 9% sucrose induction medium. The MCS are transferred from a cell strainer to a sterile container and suspended in 9% sucrose induction medium. Agrobacterium-mediated transformation of MCS cells is performed as described herein, including the preparation of the Agrobacterium master plant and growth of Agrobacterium on a solid medium as described above.

The results of this experiment indicate that microspore embryogenesis is stimulated in response to the combined Agrobacterium mixture treatment with treated paternal haploid cells transferred to 605J medium without selection after 72 hours. Several hundred to thousands of microspores treated in this manner in a highly scalable system that is either fully or partially automated. In another embodiment, the methods of the present disclosure can be practiced as described above with Agrobacterium-treated MCS transferred to a solid medium with antibiotic (e.g. 605T).

In addition, the method for transformation can be practiced as described above using the same steps through maize transformation and chromosome doubling steps and then transferring proliferating callus tissue derived from a paternal haploid cell to a maturation medium with selection, for example 289Q medium supplemented with 5-fluorocytosine. Conditional selection methods can also be practiced during or after proliferating callus tissue of each treated microspore-derived embryo is dissected. For example, by transferring each portion of dissected tissue to maturation medium (289Q) supplemented with 5-fluorocytosine and cultured at 26-28° C. under dark conditions.

### Example 9

### Methods of In Vitro Plant Breeding

Meiosis is a process for forming haploid gametes from diploid germ cells, a process essential for sexually reproducing species to transmit genetic information to the next generation. Meiosis requires a specialized cell cycle consisting of one round of DNA synthesis followed by two successive rounds of M phase that reduces the number of chromosomes in the parent cell by half After meiosis occurs, each haploid cell contains a mixture of genes inherited from the maternal and paternal genome following the principle of independent assortment. Independent assortment requires homologous pairs of chromosomes must be identified and matched, called pairing, during Meiosis I. The matched pairs must be physically interlocked by recombination, also referred to as exchange or crossing-over, resulting in meiotic recombination between two homologous chromosomes thereby producing the recombinant gametes. Methods manipulating control of a cell's regulation of mitosis-meiosis pathways are of interest to the present disclosure, specifically control of molecular mechanisms promoting a cell's commitment for entry into meiosis.

For breeding purposes, a cell containing chromosomal regions that are heterozygous can be used, wherein the desired outcome is the production of meiotic recombinant cells. The use of an F1 hybrid embryo resultant from a biparental cross is an exemplary cell type for this method. It is understood that other cell types, for example from subsequent generations or from other breeding approaches, can be generated, obtained, and used for treatment as described below.

Commitment to entry into meiosis seems to be highly conserved in eukaryotes and the process has been conceived as comprising two parts: i) exit from the mitotic cell cycle and, ii) induction of the alternate meiotic cell cycle program. As described here, the former step for inducing mitotic cell cycle activity is stimulated in response to expression of a morphogenic developmental gene, for example as described in Example 5, wherein such treatments characteristically cause a relatively high mitotic activity that results in the induction of somatic embryogenesis. Although induction of the alternate meiotic cell cycle program is less well characterized, here the present disclosure describes treatments useful for controlling a cell's commitment to entry into meiosis.

Treatments useful for entry into meiosis include, but are not limited to, responses to reducing treatments that increase hypoxia and or lower cellular hydrogen peroxide levels, by either environmental or chemical means. Cell treatments providing activity of a gene product, or gene products, useful for committing entry of the cell into meiosis are also of interest.

In the current method, exposing a cell of an F1 embryo to redox-modulatory conditions is of interest.

Exposing a cell of an F1 embryo to redox-modulatory conditions comprises transfer of the treated cell to a hypoxia-inducing tissue culture system, such as use of a hypoxia incubator chamber (StemCell™ Technologies, catalog #27310) or use of oxygen-controlling biomaterials, such as hypoxia-inducible (HI) hydrogels that can provide three dimensional hypoxic microenvironments before, during, or after the induction of somatic embryogenesis. In this method, the treated cells are cultured in conditions to alter the redox potential of cells, thereby promoting acquisition of a germ cell fate upon exiting a mitotic cell cycle.

In another aspect, methods for exposing an embryo cells to redox-modulatory conditions include treatment of a F1 hybrid embryo by contacting the cell of a F1 hybrid diploid embryo with chemical treatments before, during, or after the induction of somatic embryogenesis to manipulate cellular redox conditions, thereby promoting acquisition of a germ cell fate.

For example, the chemical treatment can be in the form of a gas, liquid, or solid, including but not limited to a redox-modulatory compound dissolved in a liquid or as a particle present in a liquid. A chemical treatment useful for promoting acquisition of a germ cell fate includes contacting the cell with a potassium iodide solution (KI, 10 mM) for up to 48 hours. Another chemical treatment useful for promoting acquisition of a germ cell fate includes contacting the cell with sodium nitroprusside (SNP, 20 μM), as well as a SNP chemical treatment while under hypoxic atmosphere conditions achieved by administering gases, such nitrogen gas (N₂) and or carbon dioxide (CO₂), for example to atmospheric levels less than 19.5 percent oxygen. In response to such treatment, the treated cell is thereby cultured with an altered acquisition of germ cell fate.

In another aspect, it is believed that meiosis-specific gene products can alter control of a cell's commitment to entry into meiosis. Thus, methods for contacting the cells with a gene, or genes, conferring functional activity for committing entry of the cell into meiosis are of interest. Such genes useful in the present method include but are not limited to ectopic expression and biological activity of A-type cyclins and/or a single B-type cyclin (CYCB3;1) shown to contribute to meiosis-related processes. Other examples of genes useful for promoting transitions into a meiotic cell cycle include ectopic overexpression of TAM (Tardy Asynchronous Meiosis, also known as CYCA1;2), OSD1 (Omission Of Second Division), TDM (Three-Division Mutant), and SMG7 (Suppressor With Morphogenetic Effects On Genitalia 7). Exemplary candidate sequences are shown in Table 18.

After a F1 hybrid embryo is isolated, mitotic cell cycle activation is initiated using methods described in Example 3 and 5, and during this process the cells can be exposed to redox-modulatory conditions described above, that can simultaneously include contacting the cells with a gene product, or gene products, useful for committing entry of the cell into meiosis (see Example 14 for details), thereby promoting entry into a meiotic cell cycle program to acquire a germ cell fate. After incubation using resting medium under such conditions, an induced embryo containing a meiotically recombined genome that is equivalent to a new haploid gamete is individually transferred onto regeneration medium (289Q).

Preferentially, meiotically recombinant cells generated by this method can be separated using vortexing to separate the germinating embryos. For example, at twenty (20) days post Agrobacterium tumefaciens infection, the developing tissue is transferred to a sterile 50 ml centrifuge tube containing approximately twenty (20) mL of liquid, vortexed, and the tissue is transferred onto regeneration medium (289Q). The embryos are cultured under dark conditions until shoot and root formation is initiated and then transferred under light conditions for plantlet regeneration.

In one aspect, dissected haploid embryos can be transferred to resting regeneration medium (605J or 289Q medium) with a chromosome doubling (or mitotic inhibitor) agent, for example colchicine at concentrations of 0.1-1.0 g/ml to cause mitotic arrest of dividing cells at metaphase by interfering with microtubule organization, for example for a 24-hour period and then transferring to a regeneration resting medium (e.g. 605J, 289Q medium, or preferentially 605T medium) without a chromosome doubling agent followed by incubation at 28° C. under dark conditions. After three (3) to seven (7) days, the embryos are transferred to maturation medium (289Q medium) without selection.

In another aspect, meiotically recombinant haploid cells can be isolated and two such cells can be fused. Methods include electrically or chemically fusing cells and such cultured products have been shown to develop into fertile plants.

After completing such a chromosome doubling treatment or a cell fusion treatment, a sub-cultured tissue is transferred to a light culture room at 26° C. until healthy plantlets with good roots develop. Approximately 7-14 days later, plantlets are transferred to soil and typically grown for one week in a growth chamber, subsequently grown an additional 1-2 weeks in the greenhouse, and then transplanted to soil and grown to maturity.

After a regenerated plant further develops, a leaf tissue sample can be collected per plant, DNA is isolated, and a diagnostic PCR-based assay is performed to detect presence/absence of a T-DNA, and or any related plasmid DNA sequence, and or to compute a genomic estimated breeding values (GEBVs) as described in Example 6.

The creation of novel genetic entities is achieved by ectopically activating meiotic recombination before, during, or after initiation of mitosis, thus providing a novel approach for performing “in vitro nursery” breeding activities. In contrast to the results shown in Example 5, wherein the resulting somatic embryos are clonal somatic embryos, here the method produces non-clonal gametic cells used to generate fertile plants useful for plant breeding.

Entry into meiosis is thought to comprise two parts, i) exit from the mitotic cell cycle and, ii) induction of the alternate meiotic cell cycle program, here the methods of the present disclosure promote entry into a meiotic cell cycle program, for example in response to mitotic cell cycle activity as shown in Example 5. Methods of stimulating entry into a meiotic cell cycle program comprising cell treatments that increase hypoxia and or lower cellular hydrogen peroxide levels, using either environmental and or chemical methods, and cell treatments providing activity of a gene product, or gene products, are expected to be useful for committing entry of the cell into meiosis and it is furthermore expected that combinations of these methods can result in greater efficacy.

Likewise, it is expected that simultaneously providing two or more gene products useful for inducing a meiotic cell cycle program, including but not limited to proteins described in Tables 18, 19 and or 20, can result in greater efficacy for inducing a meiotic cell cycle program, thereby improving the productivity of the methods disclosed herein of obtaining embryogenic cells with genomes produced by meiotic recombination.

Thus, it is expected that a treated embryo explant, such as an F1 embryo resultant from biparental fertilization between two inbred plants, can be cultured to produce embryogenic cells having genomes that are each the product of meiotic recombination. After using the methods disclosed herein in combination with other tissue culture methods, it is expected an embryogenic cell can produce a plant with a genome comprising a novel genetic entity that is useful for plant breeding.

### Example 10

### Transient Expression of Meiotic Genes for High-Throughput In Vitro Plant Breeding

The methods of stimulating entry into a meiotic cell cycle program described that include providing the activity of a gene product, or gene products, useful for committing entry of the cell into meiosis. Methods for transiently expressing such meiotic genes are now described. An Agrobacterium can be used to stimulate mitotic activity and embryogenesis in a plant cell. Here a second Agrobacterium with a plasmid comprising an expression cassette with a polynucleotide encoding a “meiosis induction” protein is used. More specifically this expression cassette can have one or more polynucleotide sequences encoding the gene products as described herein. In an aspect, the meiosis induction expression cassette can be designed for Agrobacterium-mediated protein expression, wherein the polynucleotide does not integrate into the genome of the plant and the plant does not contain vector (plasmid) backbone.

One method includes “beyond the border” plasmid designs. Another method uses Agrobacterium-mediated transient protein delivery into a plant cell and is used here specifically for delivery into a plant cell of meiosis-specific gene products as described herein. This Agro-mediated delivery of meiosis-inducing factors are automated to increase throughput of the system.

A first Agrobacterium strain is used and upon infection, this provides a T-DNA to a first plant cell that allows the first plant cell to express the WUS protein. The WUS protein can move in a non-cell autonomous manner, thereby the protein is provided to a second plant cell. In the present method, the second plant cell is not exposed to the presence of a T-DNA. Once a second plant cell is contacted with a WUS protein, it is of interest to simultaneously contact the second plant cell with a meiosis-specific gene product, or products, resulting from co-infection with a second Agrobacterium strain. Here, the second Agrobacterium strain provides translocation into the second plant cell by expressing a translational fusion protein comprising a Vir translocation peptide domain fused to another induction peptide. In the present method, a polynucleotide encoding the translational fusion protein, or proteins, is operably linked to the virF promoter and, consequently, is regulated by the virA/virG genes as part of the phenolic-induced vir regulon. Here, a virF-meiosis induction replicon introduced into an A. tumefaciens strain, for example AGL1 THY—without T-DNA borders, which is used to transiently express and ectopically deliver the meiosis induction protein(s) into a plant cell. Specifically, co-infection using two Agrobacterium strains as described above provides a combination of i) mitotic cell cyle activity and ii) induction of an alternate meiotic cell cycle program to result in the development of haploid, embryogenic cells having genomes produced by meiotic recombination.

In the automated methods of the present disclosure, an immature F1 hybrid embryo is an exemplary explant used for the following treatment, where a large-scale amount of hybrid or other immature embryo is used with the aid of automated robotic processes. Using the two Agrobacterium strains as described above, the two different strains are combined in a mixture, for example a mixture with a first Agrobacterium strain encoding the WUS protein expression cassette (Agro1) and a second Agrobacterium strain encoding the meiosis induction expression cassette (Agro2). Ratios of Agro1:Agro2 can be 95:5, or 90:10, and other ratios. For treating a cell of the F1 hybrid diploid embryo, the bacterial/plant cell ratio of approximately 1000:1 is used. After 24 hours, the plant cells are washed, and the medium replaced with medium containing Timentin to kill Agrobacterium. Preferentially, the cells can be cultured using the environmental and or chemical treatments as described herein—before, during, or after Agrobacterium co-infection. Following co-cultivation each diploid embryo exhibiting a somatic embryogenesis response is cultured and plantlets are regenerated as described herein using automated methods.

The transient expression method described herein provides a method for simultaneous co-infection of a plant explant, for example an immature F1 hybrid embryo. Such methods described herein are useful for both precociously expressing meiotic genes and for transporting such ectopic activity into a plant cell, such as the protein activities described in Example 13. It is also expected that changing the redox potential of cells can further improve the induction of a meiotic cell cycle program in response to the co-infection process described. Thus, it is expected that this method enables capabilities for creating plants derived from somatic embryos that are F2 generation equivalents, thereby providing a novel method useful for “in vitro nursery” breeding activities.

### Example 15

### Generating Wheat Clonal Doubled Haploids Using Homology-Directed Repair (HDR)

The following experiments demonstrate using a gene targeting system to facilitate directional targeting of desired genes and nucleotide sequences into corresponding homologous recombination sites on each of three paired (homologous) sets of chromosomes of a wheat (Triticum aestivum) haploid cell.

Immature wheat haploid embryos are created using methods, for example, using wide hybridization methods. Additional methods known in the art can include haploid induction resulting from expression of an AP2 domain transcription factor. For such AP2 domain transcription factor methods, Agrobacterium-mediated stable plant transformation is employed. For example, the ODP2 nucleotide sequence introduced into the plant is under the control of a tissue specific promoter that is active in a haploid cell or tissue or a promoter that is active during male or female gamete development. Alternatively, the ODP2 nucleotide sequence is under the control of an inducible promoter and the application of the inducer allows expression of the ODP2 sequence therein. Alternatively, the promoter used can be both inducible and tissue-preferred. For example, the promoter can be both haploid-tissue specific and inducible.

Preferentially, methods of the present disclosure can further use site-specific recombination systems in combination with such an ODP2 expression cassette operably linked to such promoters (i.e., constitutive promoters, tissue-specific promoters, or inducible promoters). For example, by using a first lox site and a second lox site flanking the ODP2 expression cassette, wherein the promoter is operably linked to a polynucleotide encoding the ODP2 polypeptide to be active during either male or female gamete development.

A promoter expressed in the egg cell of the plant is useful for regulating ODP2 expression to promote maternal haploid induction, resulting in a percentage of the progeny to be haploid having half the number of chromosomes compared to the parent. For example, using exemplary promoters including but not limited to AT-DDS, AT-DD31, AT-DD65, or more preferentially the ZM-DD45 promoter. Additionally, a Zea mays egg cell promoter operably linked to a polynucleotide encoding the ODP2 protein and a 3′ UTR from a Zea mays egg cell gene can be used.

The methods of the present disclosure further comprise transforming a F1 hybrid wheat embryo with Agrobacterium strain LBA4404 THY- (See U.S. Pat. No. 8,334,429 incorporated herein by reference in its entirety) as described in Example 2. Transformation of a F1 hybrid wheat embryo is performed to create a stable, hemizygous transgenic plant containing the ODP2 expression cassette operably linked to an egg cell promoter. Upon transitioning to the reproductive phase, the plant self-fertilizes, and maternal haploid embryos can be collected using available methods.

Clonal propagation of doubled haploid plants in combination with gene targeting is performed by then transforming a maternal haploid embryo using Agrobacterium transformation methods as described above. In the current method, a mixture with another Agrobacterium, here containing a gene targeting plasmid with a T-DNA comprised of multiple “Traits” (for example a zinc finger nuclease, a donor excision template encoding a trait gene, and an anthocyanin color marker;) can be used.

Transformed cells with excised donor templates can be detected using morphological detection of anthocyanins expressed by a functional B-Peru gene resulting from excision of the donor template, thereby allowing B-Peru gene regulation from the ZmGlob1 regulatory sequence. Stable transgenic plants are identified using positive selection, for example by identifying plants with herbicide resistance to haloxyfop.

It is expected that culturing such plants in the presence of a chromosome doubling agent will facilitate chromosome doubling and diploidization of the stable transgenic doubled plants.

It is expected that each hemizygous, single copy plant with herbicide tolerance will have a “Trait” gene inserted at a targeted site in the genome and the remnant “Trait” T-DNA comprising the repaired T-DNA sequence inserted at a random position in the genome.

Within a doubled haploid clone set possessing the “Trait” gene inserted at targeted sites, selecting clonal individuals for cross-fertilization is performed, thereby creating seed of this cross to be propagated. Some progeny can be expected to be wild type at loci segregating for each random T-DNA insertion site comprising the repaired “Trait” T-DNA sequence with also inheritance of alleles from both parental gametes with a successful gene targeting event at the targeted transgene integration site. Selection of an individual without the repaired “Trait” T-DNA, while possessing alleles with a successful gene targeting event at the targeted transgene integration sites can be self-fertilized. Further propagation of such plants will then segregate as clonal doubled haploids with homozygous copies of the “Trait” gene at each homologous chromosome's target site.

The methods of the present disclosure are an improvement to accelerate trait introgression using wheat haploid induction breeding methods, thus, enabling forward breeding capabilities for site-directed transgene integration in wheat doubled haploids without the need for backcrossing-mediated trait introgression.

### Example 16

### Automated Methods to Produce Non-Transgenic Somatic Embryos and Plants in Wheat

Freshly harvested wheat immature grains are sterilized with 50% bleach and 0.1% Tween-20 for thirty (30) minutes under vacuum and then rinsed with sterile water three times in a platform suitable for automated handling of hundreds to thousands of wheat embryos. Agrobacterium is used in some embodiments. After a 15-minute liquid infection treatment, the immature embryos are removed from the liquid medium and transferred onto solid medium 606 medium and oriented scutellum-side up for culture at 21° C. in the dark overnight, using automated/semi-automated mechanical devices controlled by a computer. The embryos are transferred again onto fresh resting medium (606) for ten (10) days or fresh media is replaced where the embryos are resting, then onto regeneration medium 689E with selection in the dark. The tissue is then moved onto regeneration medium 689E through an automated robot with selection in the light and then the number of plants produced is tabulated.

In a treatment, immature embryos of Spring wheat variety SBC0456D are infected with the Agrobacterium, and producing plants from scutellum-derived somatic embryos, in an automated high-throughput setting. The improvement in the final number of non-transgenic plants represented a substantial improvement in micro-propagation producing non-transgenic plants that are then ready for transfer to the greenhouse.

### Example 17

### Automated Methods to Produce Non-Transgenic Somatic Embryos and Plants in Sorghum

Agrobacterium strain LBA4404 THY is used in some treatments. After a 15-minute liquid infection treatment, the immature embryos are removed from the liquid medium and transferred onto solid medium 562V and oriented scutellum-side up for culture at 21° C. in the dark overnight. Freshly harvested sorghum immature grains are sterilized with 50% bleach and 0.1% Tween-20 for 30 min under vacuum and then rinsed with sterile water three times, in an automated fashion using mechanized platforms. The embryos are subjected to the following five sequential steps in an automated fashion: (1) Agrobacterium infection: embryos are incubated in an Agrobacterium suspension (OD=0.7 at 550 nm) with PHI-I medium for 5 min; (2) co-cultivation: embryos are cultured on PHI-T medium following infection for 3 d at 25° C. in the dark; (3) resting: embryos are cultured on PHI-T medium plus 100 mg/l carbenicillin for 4 d at 28° C. in the dark; (4) selection: embryos are cultured on PHI-U medium for 2 wk, followed by culture on PHI-V medium for the remainder of the selection process at 28° C. in the dark, using subculture intervals of 2-3 wk; (5) regeneration: callus is cultured on PHI-X medium for 2-3 wk in the dark to stimulate shoot development, followed by culture for 1 wk under conditions of 16 h light (40-120 μE m-2 s-1) and 8 h dark at 25° C., and a final subculture on PHI-Z medium for 2-3 wk under lights (16 h, 40-120 μE m-2 s-1) to stimulate root growth. Optionally, morphogenic factors are used during the embryogenesis phase. Regenerated plantlets are now ready to be transplanted into soil and grown in the greenhouse.

### Example 18

### Scalable, Automated Methods for Improving Regeneration of Amphiploid Plants Including Automated Embryo Rescue Methods

Wide hybridization is a plant breeding tool, including both interspecific and intergeneric hybridization, used to produce a hybrid from a cross of related species or genera that do not normally sexually reproduce with each other, herein referred to as a ‘wide cross’. Wide hybridization is the first step to introduce and transfer desirable trait(s) from a wild species, often referred to as an ‘alien’ species, into a cultivated species lacking a favorable phenotype for said trait(s). Methods to automate the haploid induction by wide-crosses are desirable given the haploid induction efficiencies are generally low compared to strong intraspecific haploid inducers.

Introgression has two key steps: sexual hybridization to bring the wild or ‘alien’ genome into a cultivated background and homologous and/or homoeologous recombination to eliminate or replace the deleterious alleles and/or genes causing a reduction in fitness in a cultivated species. However, deleterious genes introgressed along with the beneficial gene transferred from a wild, ‘alien’ species can occur. Thus, a major disadvantage when using wild genetic resources is that amphiploids, the first-generation hybrid containing a diploid set of chromosomes from both parents, can have reduced fitness due to deleterious genes transferred from ‘alien’ species as reported in the art.

For this reason, backcrossing is often performed, wherein an amphiploid is crossed to the cultivated species to obtain offspring with a genetic identity closer to that of the cultivated species parent. In each back-cross generation, the beneficial gene from the ‘alien’ genome is maintained, while a gene conferring an unfavorable trait(s) is selected against. Recombination is required during each generation to obtain novel recombinants to ‘break’ such linkage between genes conferring beneficial and unfavorable traits. Multiple generations of backcrossing are typically performed using the cultivated species as the ‘recurrent’ parent to obtain a plant possessing the original agronomic fitness of the cultivated species with, ideally, only the introgressed gene(s) transferred from the ‘alien’ species conferring improved phenotypic performance for said trait(s).

Thus, the phenotypic diversity of landraces, local cultivars and related species are sources of genetic variation useful for crop improvement, and thus, are useful for improving the sustainability of current agricultural production methods. A drawback in using interspecific hybridization in plant breeding programs, however, is the low probability of obtaining in one individual, the desired combination of genes from the parental species. Hence, methods that improve this probability for obtaining the desired outcome are desirable.

Plant breeding methods include creating populations such as chromosome segment substitution lines, recombinant chromosome substitution lines, near-isogenic lines, as well as chromosome addition and chromosome translocation lines.

As described herein, a Triticeae example is considered as an exemplary model, given that many synthetic wheats lines have been created using such methods and novel genetic diversity continues being incorporated into wheat breeding programs worldwide. It is also useful given that genetic material for improving such traits is described as a diverse set of Triticeae species comprising primary, secondary, and tertiary gene pools of wheat. These gene pools include wild and cultivated species within the genera Aegilops, Agropyron, Ambylopyrum, Dasypyrum, Elymus, Hordeum, Leymus, Lophopyrum, Psathyrostachys, Pseudoroegneria, Secale, Thinopyrum, and Triticum.

In relation to a cultivated species, it is believed that the evolutionary distance between two parental lines is proportional to how ‘wide’ a cross is, for example, as characterized by DNA sequence differences at the genomic level.

Difference at the genomic level are believed to be underlying factors creating barriers affecting the success of using wide cross methods, despite the potential use of existing biodiversity for genetic gain. Examples of such barriers include but are not limited to meiotic pairing characteristics in diploid hybrids, for example, a lack of chromosome pairing, preferential transmission of chromosomes harboring gametocidal genes, hybridization incompatibility due to sterility, and suppressed recombination, typically due to a lack of synteny. As a result, despite some highly significant successes, introgression remains laborious and time consuming, increasingly so as the genetic basis for trait complexity increases, and therefore it is believed that today's cultivated elite crop gene pools contain only a fraction of the available biodiversity.

Methods for overcoming such barriers include using irradiation to generate translocations and use of gametocidal genes for induced chromosomal breakage. Tissue culture improvements are also practiced.

Despite such improvements, amphiploid seed can remain defective and fail to germinate under normal conditions. Embryo rescue is used to improve regenerating a plant from an immature amphiploid embryo. When such an amphiploid embryo would otherwise perish after being allowed to further develop as a seed, automated embryo rescue methods improve the probability for obtaining an amphiploidy plant, and thus greater access to genetic diversity present in other gene pools.

The present disclosure also provides methods useful for improving or circumventing remaining barriers that restrict or prevent genetic interchange between related plant populations. Here, the present disclosure describes novel improvements, whereby amphiploid embryos are rescued and treated as described herein. For example, amphiploid embryos can be treated to further improve capabilities of regenerating amphiploid plants, including the capability to obtain more than one amphiploid plant per treated immature embryo, using one or more of the embryogenic methods described above.

In the method of the present disclosure, freshly harvested immature seed produced by a wide cross, for example resulting from a wide cross between two Triticeae species, are sterilized with 50% bleach and 0.1% Tween-20 for 30 min under vacuum and then rinsed with sterile water three times, using automated devices.

After the 15-minute liquid infection treatment, the immature embryos are removed from the liquid medium and transferred onto solid medium 606 medium and oriented scutellum-side up for culture at 21° C. in the dark overnight. The embryos are transferred again onto fresh resting medium (606) for 10 days or a fresh medium is replaced, then onto regeneration medium 689E with selection in the dark. The tissue is then moved onto regeneration medium 689E with selection in the light.

Using chromosome doubling methods with colchicine as described herein, a treated embryo exposed to a chromosome doubling agent before, during, or after the Agrobacterium co-infection steps can produce one or more first-generation hybrid plants containing a diploid set of chromosomes from both parents, or amphiploid plants. Colchicine treatments are highly automatable and scalable for doubling several hundred or thousands of wide-cross rescued embryos. After germination, the plants produced are potted to soil, and the count of clonal plants per treated embryo are recorded.

Tissue can be collected from a plant and biomolecules such as DNA, RNA, proteins, and or metabolites can be isolated for diagnostic analysis, in a high-throughput automated fashion.

Thus, in one aspect, it is expected that wide hybridization crosses can be obtained with improved efficiencies, and plant breeding programs can more effectively introgress and evaluate novel genetic variants existing in landraces, local cultivars and related species.

In another aspect, the results of the current method are expected to improve generating and selecting wide cross progeny with a reduced frequency of deleterious alleles. Such a method can be expected to reduce the preferential transmission of chromosomes harboring gametocidal genes. Thus, the method of the present disclosure is expected to improve capabilities for using wild genetic resources in wide crosses.

In another aspect, the methods of the present disclosure can be used to overcome other barriers to wide cross methods, such as barriers caused by a lack of chromosome pairing, hybridization incompatibility due to sterility, or suppressed recombination caused by a lack of synteny. It is expected that the methods described here for creating a targeted double strand break in the genome of a cultivated species genome while simultaneously providing donor template from an alien species can improve the frequency for recovering wide cross progeny having ‘alien’ introgressions. Such a capability is expected to improve methods for creating populations such as chromosome segment substitution lines, recombinant chromosome substitution lines, near-isogenic lines, as well as chromosome addition and chromosome translocation lines.

Together, the high-throughput automated methods of the present disclosure are expected to reduce the amount of time and labor required for breeding methods aiming to widen the breadth of available biodiversity within today's cultivated elite crop gene pools. When combined with other methods of plant breeding, it is furthermore expected that the methods of the present disclosure will increase the probability of obtaining in one individual the desired combination of genes from the parental species used in wide crosses.

### Example 19

### Automated Treatment, Handling, and Phenotyping Methods for Obtaining Clonal Plants

Using methods described above, methods of the current example use automation for producing clonal, non-transgenic plants. For example, when generating clonal plants as described for Examples 3-9, 15, and 18, here the methods of the present disclosure describe using automated processes before, during and after the clonal propagation steps.

For example, clonal propagation activities are conceived as being performed using an apparatus for preparing a plant tissue, to hold a tissue, for applying an Agrobacterium to a plant tissue, for transferring plant tissue, for culturing plant tissue, and or for subjecting the tissue to a force to divide the tissue into separate segments. It is expected such steps may use more than one apparatus.

Preferentially, it is conceived that the above automation steps can also comprise integrating analytical capabilities including but not limited to use of different sensors and image capture systems. For example, an apparatus may be used for acquiring an image, such as visual, hyperspectral, thermal, or fluorescence imaging. In another example, an apparatus may be used for measuring, capturing, and analyzing qualitative and quantitative traits, such as biomass, shape, thickness, volume, growth rate, and or morphological characteristics. The apparatus may also be used for measuring and analyzing of other parameters, such as light intensity, light duration, water and nutrient uptake, evaporation and transpiration components, and or quantitative measurement of the complete set of ions or changes in ion production under varied external stimuli.

It is understood the acquired images and data is for data analysis and interpretation using methods known in the art; no effort is made here to describes all methods pertinent to the current example. It is understood such methods can include performing feature extraction for classification purposes, wherein such classification can be used for predictive model generation. Such predictive modelling is a computer implemented model encompassing a variety of statistical techniques from data mining, automated feature extraction, and machine learning.

It is also expected that such data analysis comprises linking genotypic data with captured phenotypic data, including methods measuring biomolecules from a tissue before, during, or after such automation methods are performed.

It is expected that the results of the automate methods described here can be linked with genotypic data, for example to enable predicting phenotypic performance using a biological model based on genomic data of a characterized plant, tissue, or plant cell treated using methods of the present disclosure.

It is expected that the methods of the present disclosure can improve the capability for producing clonal plants, including but not limited to aspects for improving the regeneration frequency, improved transplanting success, and ultimately improvements for the reproductive success of the clonal plants produced using such automated treatment, handling, and phenotyping methods.

In another aspect, it is expected that the results of linking genotypic data with the acquired phenotypic data will enable improving predicting phenotypic performance using a biological model based on genomic data of a characterized plant, tissue, or plant cell treated.

It is furthermore expected that the methods of the present disclosure can improve plant breeding outcomes, for example when such automated treatment, handling, and phenotyping methods that co-integrate the above data analysis and interpretation with the method of selecting clonal doubled haploid plants based on a genomic estimated breeding value. It is expected this method can provide a novel capability to improve the productivity of a non-random, structured breeding population. It is expected this result can more cost-effectively provide a population with the number of individuals required to have some specified quantity of interest. Thus, in comparison to having the same probability in the idealized population, such as the effective population size required when provided a random population, the methods here are expected to improve the relative rate of genetic gain while using relatively fewer resources. As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

That which is claimed:

1. An automated, high-throughput method of producing a population of clonal plants, the method comprising:
providing to a population of first plant cells a morphogenic factor in a high-throughput and automated manner, wherein the population of first plant cells are derived from a plurality of distinct parental lines;
eliciting a growth response in a population of second plant cells, wherein a substantial portion of the population of second plant cells does not contain an exogenous polynucleotide encoding the morphogenic factor; and
regenerating the population of clonal plants from the population of second plant cells using an automated plant cell sorting and growth platform, wherein the regenerated population of clonal plants do not contain the exogenous polynucleotide encoding the morphogenic factor.

2. The method of claim 1, wherein the population of first plant cells are cells derived from one or more haploid (1n) cells.

3. The method of claim 2, wherein the haploid cells are microspores.

4. The method of claim 2, wherein the haploid cells are embryos.

5. The method of claim 1, wherein an automated, high-throughput genotyping and/or phenotyping analysis is performed after the provision of the morphogenic factor and before the regeneration of the plants.

6. The method of claim 5, wherein the genotyping and/or phenotyping is non-destructive.

7. The method of claim 1, wherein the substantial portion of the population of second plant cells are treated with a chromosome doubling agent.

8. The method of claim 7, further comprising: crossing a regenerated clonal plant from the regenerated population of clonal plants with a plant comprising a desired genotype/phenotype; and growing offspring having the desired genotype/phenotype.

9. A plant seed produced from a doubled haploid plant produced by the method of claim 1 and any progeny derived therefrom.

10. An automated method of high-throughput analysis of clonally propagated plant cells, the method comprising:
characterizing a large number of plant cells that comprise a population of second plant cells or any cell derived from the population of second plant cells that do not contain any heterologous polynucleotide associated with morphogenesis compared to a first population of plant cells that is exposed to an exogenous morphogenic factor or contain in their genome the heterologous polynucleotide encoding the morphogenic factor, wherein the characterization includes data obtained from one or more genotyping and/or phenotyping experiments in an automated, high-throughput manner wherein a sample is obtained for genotyping and/or phenotyping analysis such that the non-sampled population of cells remain viable for clonal reproduction;
predicting phenotypic performance of the second plant cells or a substantial portion thereof using a biological model based on the genotyping and/or phenotyping data of population of second plant cells that are characterized; and
selecting a second plant cell from the population of second plant cells based on the predicted phenotypic performance; and regenerating a clonal plant derived from the selected second plant cell.

11. The method of claim 10, wherein characterizing is selected from the group consisting of:
high-throughput genotyping of DNA isolated from the second plant cell or the cell derived from the second plant cell; or
high-throughput measurement or detection of RNA transcripts isolated from the second plant cell or the cell derived from the second plant cell; or
high-throughput measurement or detection of nucleosome abundance or densities of chromatin isolated from the second plant cell or the cell derived from the second plant cell; or
high-throughput measurement or detection of post-translational modifications of histone proteins of chromatin isolated from the second plant cell or the cell derived from the second plant cell; or
high-throughput measurement or detection of epigenetic modifications of DNA or RNA isolated from the second plant cell or the cell derived from the second plant cell; or
high-throughput measurement or detection of protein:DNA interactions of chromatin isolated from the second plant cell or the cell derived from the second plant cell;
high-throughput measurement or detection of protein:RNA interactions or complexes isolated from the second plant cell or the cell derived from the second plant cell; and a combination of the foregoing.

12. The method of claim 10, wherein predicting phenotypic performance is selected from the group consisting of:
using large-scale genomic data based on genotyping by DNA sequencing of the second plant cell or the cell derived from the second plant cell; or
using genomic data based on genotyping by assay of the second plant cell or the cell derived from the second plant cell; or
using large-scale genomic data based on a known or predicted expression state of the second plant cell or the cell derived from the second plant cell; or
using large-scale genomic data based on a known or predicted chromatin state of the second plant cell or the cell derived from the second plant cell; or
using large-scale genomic data based on a known or predicted epigenetic regulatory state of the second plant cell or the cell derived from the second plant cell; or
using large-scale genotype imputation of shared haplotype genomic data of the second plant cell or the cell derived from the second plant cell; or
using large-scale pedigree history data of the second plant cell or the cell derived from the second plant cell; and a combination of the foregoing.

13. The method of claim 10, wherein the second plant cell or the cell derived from the second plant cell is selected from the group consisting of callus, undifferentiated callus, immature embryos, mature embryos, immature zygotic embryos, immature cotyledon, embryonic axis, suspension culture cells, protoplasts, leaf, leaf cells, root cells, phloem cells, pollen, seeds, suspension cultures, explants, embryos, zygotic embryos, somatic embryos, embryogenic callus, meristem, somatic meristems, organogenic callus, embryos derived from mature ear-derived seed, leaf bases, leaves from mature plants, leaf tips, immature inflorescences, tassel, immature ear, silks, cotyledons, meristematic regions, cells from leaves, cells from stems, cells from roots, cells from shoots, gametophytes, sporophytes, microspores, multicellular structures (MCS), embryo-like structures; and a combination of the foregoing.

14. A clonally propagated population of plant seeds of the regenerated clonal plant produced by the method of claim 10 and a plant seed or any progeny resulting therefrom.

15. A high-throughput method of producing a plurality of transgenic plants having a single copy of a trait gene expression cassette comprising:
providing to a population of haploid embryos or embryo-like structures a trait gene expression cassette and a morphogenic gene expression cassette;
selecting a subset of haploid embryos or haploid embryo-like structures containing the trait gene expression cassette and no morphogenic gene expression cassette, in a non-destructive manner and optionally transferring the selected haploid embryos or the selected haploid embryo-like structures to another platform using a mechanical device that is controlled by a computer;
contacting the selected haploid embryos or the selected haploid embryo-like structures with a chromosome doubling agent in an automated configuration for a period sufficient to generate doubled selected haploid embryos or doubled selected haploid embryo-like structures; and
regenerating transgenic doubled haploid plants from the doubled selected haploid embryos or the doubled selected haploid embryo-like structures containing the trait gene expression cassette and no morphogenic gene expression cassette.

16. The method of claim 15, wherein the providing to the population of haploid embryos or the embryo-like structures comprises particle gun delivery of the trait gene expression cassette and the morphogenic gene expression cassette.

17. The method of claim 15, wherein the providing to the population of haploid embryos or the embryo-like structures comprises simultaneously contacting the population of haploid embryos or the embryo-like structures with the trait gene expression cassette and the morphogenic gene expression cassette.

18. The method of claim 15, wherein the providing to the population of haploid embryos or the embryo-like structures comprises sequentially contacting the population of haploid embryos or the embryo-like structures with the trait gene expression cassette and the morphogenic gene expression cassette.

19. The method of claim 15, wherein the providing to the population of haploid embryos or the embryo-like structures comprises bacterial-mediated delivery of the trait gene expression cassette and the morphogenic gene expression cassette.

20. The method of claim 15, further comprising:
crossing a subset of the regenerated transgenic doubled haploid plants with a population of plants comprising one or more desired genotype/phenotype in a breeding program; and
selecting offspring having the desired genotype/phenotype, wherein the selecting of the offsprings is achieved by genome prediction or another predictive process.
